(19) United States
(12) Patent Application Publication
(10) Pub. No.: US 2013/0008790 A1
(43) Pub. Date: Jan. 10, 2013

(54) COMPOSITE COMPOSITIONS FOR ELECTROPHORESIS

(75) Inventors: Timothy Updyke, Temecula, CA (US); Ilana Margalit, Ramat Gan (IL); Michael Thacker, San Diego, CA (US)

(73) Assignee: LIFE TECHNOLOGIES (ISRAEL) LTD., Carlsbad, CA (US)

(21) Appl. No.: 13/490,387

(22) Filed: Jun. 6, 2012

Related U.S. Application Data
(63) Continuation of application No. 10/946,472, filed on Sep. 20, 2004, now abandoned.
(60) Provisional application No. 60/560,310, filed on Apr. 6, 2004.
(60) Provisional application No. 60/508,786, filed on Oct. 2, 2003.
(60) Provisional application No. 60/504,683, filed on Sep. 19, 2003.

(51) Int. Cl. B01D 57/02; C07K 1/26

(52) U.S. Cl. 204/469; 204/606

(57) ABSTRACT

The invention is drawn to composite agarose/acrylamide compositions and gels. In particular it relates to gels for the separation of molecules, particularly macromolecules such as proteins. The invention is also directed to the preparation of composite gels, the separation of molecules by techniques such as electrophoresis using such gels, and the transfer of proteins from such gels to a transfer membrane using an immunoblot transfer gel.

### CROSS-REFERENCE TO RELATED APPLICATIONS

This application claims the benefit of U.S. provisional patent application Ser. No. 60/504,683, filed Sep. 19, 2002; Ser. No. 60/508,786, filed Oct. 2, 2003; and Ser. No. 60/560,310, filed Apr. 6, 2004; the disclosures of which are incorporated herein by reference in their entireties.

### FIELD OF THE INVENTION

The invention is drawn to composite gel compositions. In particular it relates to gels for the separation of molecules, particularly macromolecules such as proteins. The invention is also concerned with the preparation of composite gels, and the separation of molecules by techniques such as electrophoresis using such gels.

### BACKGROUND

This background summary is not meant to be complete but is provided only for understanding of the invention that follows. The citation of any reference herein should not be construed as an admission that such reference is available as “Prior Art” to the instant application. All patents and publications mentioned in the specification are hereby incorporated by reference to the same extent as if each individual patent and publication was specifically and individually indicated to be incorporated by reference.

Methods for separating (resolving) mixtures of macromolecules have applications such as scientific analysis (of, by way of non-limiting example, mixtures of proteins, as occurs in the field of proteomics), preparative techniques, diagnostic methods, regulatory analysis and the like. One non-limiting example of a method of resolving macromolecules (such as, by way of non-limiting example, nucleic acids, polypeptides and proteins) is electrophoresis.

Electrophoresis is a preparative and/or analytical method used to separate and characterize macromolecules. It is based on the principle that charged particles migrate in an applied electrical field. If electrophoresis is carried out in solution, molecules are separated according to their surface net charge density. If carried out in semisolid materials (gels), however, the matrix of the gel adds a sieving effect so that particles migrate according to both charge and size. Protein electrophoresis can performed in the presence of a charged detergent like sodium dodecyl sulfate (SDS) which coats, and thus equalizes the charges of, most proteins, so that migration depends on size (molecular weight). Proteins are often separated in this fashion, i.e., SDS-PAGE (PAGE refers to polyacrylamide gel electrophoresis). In addition to SDS, one or more other denaturing agents, such as urea, can also be included in order to minimize the effects of secondary and tertiary structure on the electrophoretic mobility of proteins. Such additives are typically not necessary for nucleic acids, which have a similar surface charge irrespective of their size and whose secondary structures are generally broken up by the heating of the gel that happens during electrophoresis.

In general, electrophoresis gels can be either in a slab gel or tube gel form. For slab gels, the apparatus used to prepare them usually consists of two glass or plastic plates with a space disposed between them by means of a spacer or gasket material along three sides, and the apparatus is held together by a clamping means so that the space created is open at one end. The assembly is held upright so that the open end is at the top, and a solution of unpolymerized gel-monomer is poured into the space while in its liquid state. A means of creating wells or depressions in the top of the gel (such as a comb) in which to place samples is then placed in the space. The gel-monomer solution is then polymerized and becomes a solid gel. After polymerization is complete, the comb device is removed, the gasket at the end opposite the comb device is removed, and the gel, while still held within the plates, is then ready for use Examples of such apparatus are well known and are described in U.S. Pat. Nos. 4,337,131 to Vesterberg; 4,339,327 to Tyler; 3,980,540 to Hoefer et al.; 4,142,960 to Hahn et al.; 4,560,459 to Hoefer; and 4,574,040 to Delony et al. Tube gels are produced in a similar manner, however, instead of glass or plastic plates, glass capillary tubing is used to contain the liquid gel.

Two commonly used media for gel electrophoresis and other separation techniques are agarose and polyacrylamide. Each of these is described in turn as follows. In standard PAGE technology, gels commonly range between about 5% to about 22.5% T (T=total amount of acrylamide or other gelling agent), mostly between about 7.5 and about 15% T. Lower percentages may be employed with linear polyacrylamide. In agarose gel electrophoresis, concentrations between about 0.2-2% T may be employed.

### Agarose

Agarose is a colloidal extract prepared from seaweed. Different species of seaweed are used to prepare agarose; commercially available agarose is typically prepared from genera including, but not limited to, Gracilaria, Gelidium, and Pterocladia. It is a linear polysaccharide (average molecular mass of about 12,000) made up of the basic repeat unit agarobiose, which comprises alternating units of galactose and 3,6-anhydrogalactose. Agarose contains no charged groups and is thus useful as a medium for electrophoresis.

Agarose gels have very large “pore” size and are used primarily to separate large molecules, e.g., those with a molecular mass greater than about 200 kilodaltons (kD). Agarose gels can be prepared, electrophoresed (“run”) and processed faster than polyacrylamide gels, but their resolution is generally inferior. For example, for some macromolecules, the bands formed in agarose gels are “fuzzy” (diffuse). The concentration of agarose typically used in gel electrophoresis is between from about 1% to about 3%.

Agarose gels are formed by suspending dry agarose in an aqueous, usually buffered, media, and boiling the mixture until a clear solution forms. This is poured into a cassette and allowed to cool to room temperature to form a rigid gel.

### Polyacrylamide

Acrylamide polymers are used in a wide variety of chromatographic and electrophoretic techniques and are used in capillary electrophoresis. Polyacrylamide is well suited for size fractionation of charged macromolecules such as proteins and nucleic acids (e.g., deoxyribonucleic acids, a.k.a. DNA, and ribonucleic acids, a.k.a. RNA).

The creation of the polyacrylamide matrix is based upon the polymerization of acrylamide in the presence of a crosslinker, usually methylenebisacrylamide (bis, or MBA). Upon the introduction of catalyst, the polymerization of acrylamide and methylene bisacrylamide proceeds via a free-radical mechanism. The most common system of catalytic initiation involves the production of free oxygen radicals by ammonium persulfate (APS) in the presence of the tertiary aliphatic amine N,N,N′,N′-tetramethylethylenediamine (TEMED).

As polyacrylamide is formed during the polymerization of a mixture of acrylamide and a cross-linker, N,N′-methylenebisacrylamide, the gel contracts.

Polyacrylamide is a medium for PAGE, but requires % T greater than or equal to about 3% in order to retain its structure. That is, in general, a threshold concentration of polyacrylamide of more than about 4% is necessary for it to support its own weight.

In the case of acrylamide, various chemical polymerization systems may be used. For example, TEMED and persulfate may be added to provide polymerization initiation. Once the temperature becomes stable or approaches ambient temperature, the polymerization is assumed to be complete. If desired, an acrylamide gradient may be developed by successively adding solutions with increasing amounts of acrylamide and/or cross-linking agent. Alternatively, differential initiation may be used, so as to provide varying degrees of polymerization and thus prepare a gradient gel.

In the early 1960s, polyacrylamide gels were also polymerized by light (“photopolymerized”), using riboflavin or its more soluble derivative, riboflavin phosphate. However, these gels also required hours to polymerize, were also oxygen-sensitive, and the polymerization reaction was no more reliable than the chemically-polymerized system. Riboflavin-initiated (riboflavin photolytically degrades and is thus not a catalyst per se) systems have fallen into disuse, and citations of riboflavin-polymerized gels in the scientific literature are now only historical.

Non-limiting examples of polyacrylamide-agarose compositions have been reported (U.S. Pat. No. 5,785,832 to Chiari et al., entitled “Covalently Cross-Linked, Mixed-Bed Agarose-Polyacrylamide Matrices for Electrophoresis and Chromatography”; Andrews, “Electrophoresis on Agarose and Composite Polyacrylamide-Agarose Gels”, Electrophoresis, Clarendon Press, pg. 148-177 (1986); Bates et al., “Autonomous parvovirus LuIII encapsidates equal amounts of plus and minus DNA strands” J. Virol. 49:319-324 (1984); Dahlberg et al., “Electrophoretic Characterization of Bacterial Polyribosomes in Agarose-Acrylamide Composite Gels”, J Mol. Biol. 41:139-147 (1969); Fisher et al., “Role of Molecular Conformation in Determining the Electrophoretic Properties of Polynucleotides in Agarose-Acrylamide Composite Gels”, Biochemistry 10:1895-1899 (1971); Horowitz et al., “Electrophoresis of Proteins and Nucleic Acids on Acrylamide-Agarose Gels Lacking Covalent Cross-Linkings”, Anal. Biochem. 143:333-340 (1984); Isono et al., “Lack of ribosomal protein S1 in Bacillus stearothermophilus” Proc Natl Acad Sci USA 73:767-770 (1976); Peacock et al., “Molecular Weight Estimation and Separation of Ribonucleic Acid by Electrophoresis in Agarose-Acrylamide Composite Gels,” Biochemistry 7:668-674, (1968); Rashid et al., “Electrophoretic Extraction-Concentration of Ribonucleic Acid from Agarose-Acrylamide Composite Gels”, Anal Biochem 127:334-339 (1982); Ringborg et al., “Agarose-Acrylamide Composite Gels for Microfractionation of RNA”, Nature 220:1037-1039 (1968).

### SUMMARY OF THE INVENTION

The invention is drawn to composite gel compositions. In particular it relates to gels for the separation of molecules, particularly macromolecules such as proteins. The invention is also concerned with the preparation of composite gels, and the separation of molecules by techniques such as electrophoresis using such gels.

More specifically, the present invention involves the use of a combination of synthetic monomers that can be polymerized using a free-radical based system, a cross-linker, agarose, slow-ion buffer, and a photocatalyst or photoinitiator, such as benzoin ethers, and benzophenone derivatives and an amine transfer agent, which initiates free-radical cross-linking when exposed to a source of UV light. Agarose is used to stabilize the matrix without affecting its sieving nature, and allows the solution to solidify before cross-linking takes place. Although agarose is itself a sieving material, it forms a gel with relatively large pores, whereas polyacrylamide forms gel with relatively small pores, making polyacrylamide the effective sieving entity when polymerized in the presence of agarose. By replacing the typically-used APS/TEMED system with the above system, the variance of voltage gradient across distance is minimized, resulting in the homogenous separation of sample lanes in multiple rows on the gel. The addition of an intermediate, migrating ion from the zwitterionic buffering agent N,N-bis(2-hydroxyethyl)-2-aminoethanesulfonic acid (BES) results in both sharper bands and equal running distances. BES was introduced into the buffer formulation to act as a destacking or resolving trailing ion, which, unlike the slow moving “stacking-ion” tricine in the continuous buffer formulation of Updyke et al. (see, for example, U.S. Pat. Nos. 5,578,180, 5,922,185, 6,059,948, 6,096,182, 6,143,154, and 6,162,338) or Cabilly et al. (see, for example, U.S. Pat. No. 6,562,213, and published PCT applications WO 02/18901 and WO 02/071024), is capable of resolving SDS-protein complexes in very low sieving gels.

In another aspect, the invention relates to composite gels formatted with 96-wells or 48-wells, and optionally additional wells for markers. In some embodiments the wells in successive lanes are staggered from the wells in adjacent lanes.

In another aspect, a composite gel provided herein is used in a method for separating polypeptides, wherein a polypeptide sample is loaded into the gel and an electrophoretic field is generated through the gel such that a polypeptide within the polypeptide sample migrates through the gel by electrophoresis, wherein the polypeptide sample is loaded at approximately a right angle to the gel, and wherein the gel is positioned horizontally during electrophoresis.

In yet another aspect, the invention relates to membranes and filters for use in blotting that are pre-cut to match the size and shape of pre-cast composite gels of the present invention, and kits in which such pre-cut membranes and filters are supplied with the pre-cast composite gels.

In another embodiment, provided herein is a kit that includes a separation gel according to the present invention. In illustrative examples, the separation gel is a pre-cast gel. The kit can further include the following:

one or more sample loading buffers;

one or more protein standards;

one or more pre-cut membranes for use in blotting, said pre-cut membranes having a length and a width, wherein the pre-cut membranes are pre-cut to match the length and width of the pre-cast gel;

and/or one or more immunoblot transfer gels for use in blotting, optionally having a length and a width that matches the length and width of the pre-cast gel.

Other features and advantages of the invention will be apparent from the following detailed description and claims.

### BRIEF DESCRIPTION OF THE DRAWINGS

FIG. 1 is a technical drawing showing the specifications of an exemplary gel of the invention.

FIGS. 2A and 2B are drawings showing features of a gel of the invention.

FIG. 3 shows detection of MagicMark™ Unstained Protein Standard on a gel by staining with SimplyBlue™ SafeStain (lane A), or by western blotting followed by chemiluminescent (lane B) or chromogenic (lane C) detection.

FIG. 4 shows the apparent molecular weights for E-PAGE™ SeeBlue® Pre-Stained Protein Standard.

FIG. 5 shows the results of an experiment wherein MagicMark™ protein standards were electrophoresed on a gel of the invention cast in a 96-well “staggered” format.

FIG. 6 shows the results of an experiment in which Magic Mark™ Standard was electrophoresed on a gel of the invention cast in a 96-well “staggered” format and detected by binding of antibodies in a Western blot.

FIG. 7 shows a Mother E-Base™.

FIG. 8 shows a Mother E-Base™/Daughter E-Base™.

FIG. 9 shows an EPAGE gel being loaded onto an E-Base unit.

FIG. 10 shows an EPAGE-96 gel before use.

FIG. 11 shows that the wells of the E-PAGE™ 96 Gel are staggered to provide maximum run length.

FIG. 12 illustrates that the position of the first tip should be set approximately 1 mm above the slope of the 1 well to ensure that the remaining tips are aligned above the slopes of the remaining wells.

FIGS. 13A and 13B illustrate the opening of the cassette after electrophoresis.

FIG. 14 shows results obtained using a 6% E-PAGE™ 96 Gel to resolve E-PAGE™ SeeBlue® Pre-stained Protein Standard; the gel was electrophoresed for 14 minutes.

FIG. 15 shows a photograph of one embodiment of the E-Holder™ of the present invention.

FIG. 16 shows a side view of an immunoblot assembly. The assembly comprises an immunoblot transfer gel or pad of the present invention, overlaying an acrylamide/agarose separation gel of the present invention, which in turn overlies a transfer membrane.

### DETAILED DESCRIPTION

The invention is directed to composite compositions comprising polyacrylamide and agarose “agaraose-polyacrylamide compositions,” particularly those wherein the polyacrylamide has been photopolymerized or otherwise polymerized by means that involve photolytically or photocatalytically produced free radicals.

In one aspect, the invention provides a composite composition that has a low concentration of acrylamide mixed with agarose, wherein the acrylamide has been polymerized using a photoinitiator. For example, the present invention provides a composition comprising agarose, polyacrylamide and a photoinitiator. The composition can further include one or more components such as, but not limited to, one or more salts, one or more ions, and one or more denaturants.

In certain illustrative embodiments, the composite compositions of the present invention are a gel (i.e., in a gel format), which can be a separation gel (i.e. a gel used to separation macromolecules such as proteins using electrophoresis). Accordingly, provided herein is a gel that includes agarose, polyacrylamide and a photoinitiator. The gel can be, for example, an electrophoretic gel. In one aspect, the gel further includes BES. In one preferred embodiment, the agarose is present at a concentration of between 1% and 2% and/or the BES is present at a concentration of between 10 mM and 250 mM in the gel. In certain examples illustrated herein, the gel has a first layer, a second layer, and a third layer. Each layer can include agarose and polyacrylamide. In certain aspects, the second layer also includes a photoinitiator. In one illustrative example, the gel is an E-PAGE™ 96 Gel substantially or identically as disclosed herein.

The gel, in certain examples includes a low concentration of acrylamide. By “low concentration of acrylamide” it is meant that the concentration of acrylamide is from about 0.0001% to about 25%, including, by way of non-limiting example, about 0.001%, about 0.01%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 19.5%, about 20%, about 20.5%, about 21%, about 21.5%, about 22%, about 22.5%, about 23%, about 23.5%, about 24%, about 24.5%, or about 25%.

Agarose is typically present in a composition and separation gel provided herein at a concentration of between 0.5% and 15%, 1% and 10%, 2% and 6%, 3% and 5% or in certain illustrative examples, 4%. Typically, the agarose is an ultrapure agarose. For example, the agarose can be agarose D-5 (Hispanagar, S.A., Burgos, Spain).

In certain illustrative embodiments of the invention, the gel is a pre-cast gel. A pre-cast gel is a gel that is prepared by a first party, such as a provider, and delivered to a second party, such as a customer, typically for consideration. For example, pre-cast gels in a wide variety of formats can be purchased from commercial vendors (e.g., Invitrogen). In other words, precast electrophoresis gels are typically manufactured by an outside vendor and then shipped to the laboratory where the electrophores is will be performed. In one example, the gel is a precast. E-PAGE™ 96 Gel substantially or identically as disclosed herein.

In another embodiment, provided herein is a method of resolving macromolecules, comprising subjecting the macromolecules to electrophoresis through a gel according to the present invention. The macromolecules in certain illustrative examples, are proteins. The method can be performed, for example, such that electrophoresis is carried out for between 5 minutes and 1 hour, in certain examples, between 1.5 minutes and 30 minutes. In one aspect, electrophoresis is carried out for about 15 minutes. In another aspect, electrophoresis is carried out for about 30 minutes.

The electrophoretic gels used in the invention based on polyacrylamide, also referred to herein as “separation gels” in embodiments related to immunoblotting, are produced by co-polymerization of monoolefinic monomers with di- or polyolefinic monomers. The co-polymerization with di- or polyfunctional monomers results in cross-linking of the polymer chains and thereby the formation of the polymer network. As monoolefinic monomers used in the invention can be the mentioned acrylamide, methacrylamide and derivatives thereof such as alkyl-, or hydroxyalkyl derivates, e.g. N,N-dimethylacrylamide, N-hydroxypropylacrylamide, N-hydroxymethylacrylamide.

The di- or polyolefinic monomer is preferably a compound containing two or more acryl or methacryl groups such as e.g. methylenebisacrylamide, N,N′-diallyltartardiamide, N,N′-1,2-dihydroxyethylene-bisacrylamide, N,N-bisacrylyl cystamine, trisacryloyl-hexahydrotriazine. In a broader sense, “polyacrylamide gels” also include gels in which the monoolefinic monomer is selected from acrylic- and methacrylic acid derivatives, e.g., alkyl esters such as ethyl acrylate and hydroxyalkyl esters such as 2-hydroxyethyl methacrylate, and in which cross-linking has been brought about by means of a compound as mentioned before. Further examples of gels based on polyacrylamide are gels made by co-polymerization of acrylamide with a polysaccharide substituted to contain vinyl groups, for example allyl glycidyl dextran as described in EP 87995. The gels used in the invention are prepared from an aqueous solution containing 2-40% (w/w), preferably 3-25% (w/w) of the monomers mentioned above. The amount of cross-linking monomer is about 0.5% to about 15%, preferably about 1% to about 7% by weight of the total amount of monomer in the mixture.

In addition to the initiator and monomers the reaction mixture may contain various additives, the choice of which will depend on the particular electrophoretic technique contemplated. Thus, for isoelectric focusing a certain type of ionizable compounds are added which will create a pH gradient in the gel during electrophoresis.

In another aspect, the invention relates to polyacrylamide gels of the composite composition described above, further comprising 48 wells and 4 additional wells for markers (“48-well format”). In yet another aspect, the invention relates to polyacrylamide gels of the composite composition described above, wherein the sample loading wells in successive lanes are staggered (offset) from each other, as disclosed in U.S. Pat. No. 6,562,213. In some embodiments, gels comprise 96 wells and 8 additional wells for markers (“96-well format”). Composite composition polyacrylamide gels of the present invention, in either the 96-well or 48-well formats, may be formed at concentrations of acrylamide from about 0.0901% to 25%, for example 6%, 8%, 10% or 12%.

In one aspect, a composite gel provided herein is used in a method for separating polypeptides, wherein a polypeptide sample is loaded into the gel and an electrophoretic field is generated through the gel such that a polypeptide within the polypeptide sample migrates through the gel by electrophoresis, wherein the polypeptide sample is loaded at approximately a right angle to the gel, and wherein the gel is positioned horizontally during electrophoresis.

Because photopolymerization generates less heat than other methods, the components of the composition, particularly polyacrylamide, are degraded less during this step and are generally more stable. The composition is especially suitable for electrophoretic applications and accordingly, for convenience, the invention is described with reference to electrophoresis. It is to be understood, however, that the compositions and processes of the present invention are not so limited.

Compositions and gels of the present invention include a photoinitiator and typically an amine transfer agent such as triethylamine (1-50 mM) or N-methyl diethanolamine (1-50 mM). Suitable photoinitiators, which are in some instances photocatalysts that repeatedly generate catalysts, are known in the art and include, by way of non-limiting example, the following:

Benzoin ethers, benzophenone derivatives and amines, phenanthrenequinones and amines, naphthoquinones and amines, methylene blue and toluene sulfinate (EP 0 169 397; the use of the latter two compounds for photopolymerization of polyacrylamide gels is also described by Lyumbimova et al., Electrophoresis 14:40-50, 1993);

DMPAP (2,2-dimethoxy-2-phenyl-acetophenone) and related compounds as disclosed in U.S. Pat. Nos. 3,715,293 and 3,801,329, both to Sandner et al. These patents disclose acetophenones di- or tri-substituted at the 2 position, as improvements over acetophenones substituted at the 3, 4 and/or 440 position, analogous xanthophenones, and benzoin and its lower alkyl derivatives;

Phenones, including certain acetophenones, xanthones, fluoroenones, and anthroquinones, in combination with certain amines, for example triethanolamine, are used for rapid photopolymerization of unsaturated compounds, including acrylamide, as described in U.S. Pat. No. 3,759,807 to Osborn and Tercker;

Benzophenones with benzoylcyclohexanol, as described in U.S. Pat. No. 4,609,612 to Berner et al.;

Carboxylated analogs of “Mitchler's ketone”, a diaminobenzophenone, which are water soluble photoinitiators and are described in U.S. Pat. No. 4,576,975 to Reilly; and

Photoinitiators described in U.S. Pat. Nos. 5,916,427 and 6,197,173, both to Kirkpatrick.

In preferred embodiments, the photoinitiator is selected from the group consisting of:

1-hydroxy-cyclohexyl-phenyl-ketone (1-HCPK), a.k.a. 1-hydroxycyclohexyl)phenyl-methanone, CAS Reg. No. 947-19-3 [commercially available as IRGACURE® 184 from Ciba-Geigy (Basel, Switzerland) and as SarCure SR1122 from Sartomer (Exton, Pa.)];

2,2-dimethoxy-2-phenylacetophenone (commercially available as IRGACURE® 651 from Ciba-Geigy);

2-methyl-1-[(4-(methylthio)phenyl]-2-(4-morpholinyl)-1-propanone (commercially available as IRGACURE® 907 from Ciba-Geigy);

2-hydroxy-2-methyl-1-phenyl-1-propanone, CAS Reg. No. 7473-98-5 (commercially available as DAROCUR® 1173 from Ciba-Geigy);

4-(2-hydroxyethoxy)phenyl]-2-(hydroxy-2-propyl)ketone, CAS Reg. No. 106797-53-9 (commercially available as IRGACURE® 2959 from Ciba-Geigy); and

SR1129 photoinitiator, commercially available from Sartomer (Exton, Pa.),

Other suitable photoinitiators can be used to practice the invention. See, for example, Anon., Photoinitiators for UV Curing: Key Products Selection Guide, Ciba Specialty Chemicals, Basel, Switzerland, 2002; Misev et al., Weather Stabilization and Pigmentation of UV-Curable Powder Coatings, Journal of Coatings Technology, issue of July/August, pages 34-41, 1999; and references cited in these references.

The initiators used in the present invention are preferably water soluble and can be mixed directly with the aqueous monomer solution in an amount of from about 0.1 ;M to about 250 ;M, that is, by way of non-limiting example, from about 0.5 ;M to about 50 ;M, from about 0.5 ;M to about 25 ;M, from about 1 ;M to about 10 ;M, from about 0.1 ;M to about 10 ;M, from about 0.5 ;M to about 5 ;M, about 0.1 ;M, about 0.2 ;M, about 0.5 ;M, about 0.75 ;M, about 1 ;M, about 2 ;M, about 5 ;M, about 7.5 ;M, about 10 ;M, about 15 ;M, about 25 ;M, about 40 ;M, about 50 ;M, about 60 ;M, about 75 ;M, about 90 ;M, about 1.00 ;M, about 125 ;M, about 150 ;M, about 175 ;M, about 190 ;M or about 200 ;M.

The polymerization of the monomer solution is achieved by irradiating the solution with ultraviolet light. Any light source that will activate the initiators can be used. Preferred are light sources emitting light with a wavelength within a range from about 100 nm to about 500 nm. That is, by way of non-limiting example, from about 100 nm to about 500 nm, from about 150 nm to about 450 nm, from about 200 nm to about 400 nm, from about 300 nm to about 400 nm, from about 300 nm to about 450 nm, or from about 300 nm to about 400 nm.

A suitable amount of irradiation is generally from about 0.1 joule/cm2 to about 100 joule/cm2, that is, by way of non-limiting example, from about 0.2 joule/cm2 to about 100 joule/cm2, from about 0.1 joule/cm2 to about 75 joule/cm2, from about 0.5 joule/cm2 to about 75 joule/cm2, from about 1 joule/cm2 to about 50 joule/cm2, from about 1 joule/cm2 to about 25 joule/cm2, or from about 0.5 joule/cm2 to about 10 joule/cm2.

Typically, compositions and gels of the present invention include a buffer. Any suitable buffer can be used to practice the invention. For example, the buffer can be a slow-ion buffer.

Typically, compositions and gels of the present invention include a buffer. Any suitable buffer can be used to practice the invention. For example, the buffer can be a slow-ion buffer. [0060] Typically, compositions and gels of the present invention include a buffer. Any suitable buffer can be used to practice the invention. For example, the buffer can be a slow-ion buffer. In one illustrative aspect, the buffer serves the function of an electrolyte system and therefore, provides a high buffer capacity and low conductivity. This type of electrolyte system, as disclosed in U.S. Pub. Pat. App. 20020134680 A1, entitled “Apparatus and method for electrophoresis,” Cabilly et al. is characterized by its ability to resist large changes in solution composition while keeping low current values. The high capacity and low conductivity is achieved by using pH conditions where a substantial amount of the molecules are in a non-charged form.

The use of this type of electrolyte solution eliminates the need for large reservoir tanks and allows for a small volume of electrolyte solution to be used.

The electrolyte solution enables performance of electrophoresis at a voltage of 1-50 V/cm, with conductivity of 30×10−5−140×10−5 ohm-1/cm at relatively high electrolyte concentrations, while keeping the pH in the running gel constant throughout the electrophoresis period. Electrolyte concentration may vary from 50-300 mM. In a preferred embodiment, the electrolyte concentration is 175 mM. In another embodiment, the electrolyte concentration is 100 mM.

In an illustrative embodiment, a combination of amine molecules and “Zwitterions” (ZI), also known as ampholytes, are used. These elements are combined in solution at a pH value that is higher than the pK of the amine and lower than the higher pK value of the ZI. Under these conditions the concentration of charged amine molecules and the concentration of net negatively charged ZI is low, as shown in the examples hereinbelow.

In one aspect the buffer, included in a gel of the present invention, includes an electrolyte solution comprising a weak acid and a ZI in conditions such that the pH of the solution is higher than that of the ZI and lower than the acid pK. An example of this system is a buffer at pH 4.0, composed of acetate (which has a pK of 4.72 at 25 degrees), and beta alanine (which has a pK of 3.59).

In certain illustrative examples wherein the gels provided herein are used for SDS-PAGE, the buffer includes one or more of bistris, tricine, BES, MOPS (3-[(N-Morpholino]propanesulfonic acid), or MES (2-(N-Morpholino)ethanesulfonic acid). The buffer when used to make an SDS PAGE gel, is prepared, for example, at a pH between 5.5 and 7.5, or at a pH of between about 6.5 and 9.5. In one aspect, the buffer is a neutral pH gel. In another aspect, the buffer has a pH of between 8.6 and 9.0. For alkaline pH ranges a number of exemplary buffers can be used including Bis-Tris and TAPS can be used in one example (See U.S. Pat. App. No. 2002/0134680).

Other non-limiting examples of buffers that can be used in the gels and compositions of the present invention include those described herein and in the following:

U.S. Pat. No. 5,578,180, to Engelhorn et al., entitled “System for pH-Neutral Longlife Electrophoresis Gel”;

U.S. Pat. Nos. 5,922,185; 6,059,948; 6,096,182; 6,143,154; 6,162,338, all to Updyke et al.; published U.S. Patent Applications 20030127330 A1 and 20030121784 A1; and published PCT Application WO 95/27197, all entitled “System for pH-Neutral Stable Electrophoresis Gel”;

U.S. Pat. No. 6,057,106, to Updyke et al., and published PCT application WO 99/37813, both entitled “Sample Buffer and Methods for High Resolution Gel Electrophoresis of Denatured Nucleic Acids”;

U.S. Pat. No. 6,562,213 to Cabilly et al., and published PCT application WO 02/18901, both entitled “Electrophoresis Apparatus for Simultaneous Loading of Multiple Samples”;

Published U.S. Patent Application 20020134680 A1, to Cabilly et al., and published PCT application WO 02/071024, both entitled “Apparatus and Method for Electrophoresis”; and

U.S. Pat. No. 5,785,832, to Chiari et al., entitled “Covalently Cross-Linked, Mixed-Bed Agarose-Polyacrylamide Matrices for Electrophoresis and Chromatography.”

The buffer can also include an ion exchange matrix, including a cation exchange matrix and an anion exchange matrix, collectively referred to as the ion exchange matrices (as disclosed in U.S. Pat. No. 5,865,974, Cabilly et al., “Apparatus and method for electrophoresis”). The volume of the ion exchange matrices is typically smaller than the volume of the separation gel. The cation exchange matrix and the anion exchange matrix release the cations, and anions required for driving electrophoresis separation. A suitable cation exchange material in one example of the invention, is CM-25-120 Sephadex, and a suitable anion exchange material, for example, is WA-30 and the A-25-120, all of which are commercially available from Sigma Inc. (St. Louis, Mo.).

The invention is exemplified herein with regards to gel electrophoresis of macromolecules for analysis, purification or other manipulations thereof. The electrophoretic separation is performed by conventional methods according to the specific method, use, format or application.

In certain illustrative examples related to electrophoresis, the compositions and gels provided herein, further include a charged denaturing agent. The charged denaturing agent affects the charge density of a biomolecule such as a protein being subjected to electrophoresis and affects its rate of migration through the gel—the higher the charge density, the more force will be imposed by the electric field upon the macromolecule and the faster the migration rate subject to the limits of size and shape. In SDS-PAGE electrophoresis the charge density of the macromolecules is controlled by adding sodium dodecyl sulfate (“SDS”) to the system. SOS molecules associate with the macromolecules and impart a uniform charge density to them substantially negating the effects of any innate molecular charge.

Accordingly, in one illustrative embodiments, the composite compositions and gels provided herein further include SDS. For example, the SDS can be included at a concentration typically used for SDS-PAGE such as between 0.005% SDS and 0.5% SDS, for example 0.03 to 0.07% SDS, even more specifically, for example, 0.05% SDS. In another aspect, the SDS concentration is between 0.005% and 0.1%, for example, between 0.01% and 0.1%. In another illustrative aspect, the SDS concentration is between 0.1% and 0.3%, for example 0.2%. In certain aspects, the denaturant used is lithium salt of dodecylsulfate (LDS), urea, or thiourea along with SDS or LSD. For Urea the concentration ranges in certain aspects are from about 0.5M to 10 M, typically between 8M and 5M. For thiourea the concentration range is typically between 0.5M and 3M used in combination with urea between 5M and 7M, preferably.

The gel-based electrophoretic embodiments of the invention can be carried out in any suitable format, e.g., in standard-sized gels, minigels, strips, gels designed for use with microtiter plates and other high throughput (HTS) applications, and the like. Minigel and other formats include without limitation those described in the following patents and published patent applications: U.S. Pat. No. 5,578,180, to Engelhorn et al., entitled “System for pH-Neutral Longlife Electrophoresis Gel”; U.S. Pat. Nos. 5,922,185; 6,059,948; 6,096,182; 6,143,154; 6,162,338, all to Updyke et al.; published U.S. Patent Applications 20030127330 A1 and 20030121784 A1; and published PCT Application WO 95/27197, all entitled “System for pH-Neutral. Stable Electrophoresis Gel”; U.S. Pat. No. 6,057,106, to Updyke et al., and published PCT application WO 99/37813, both entitled “Sample Buffer and Methods for High Resolution Gel Electrophoresis of Denatured Nucleic Acids”; U.S. Pat. No. 6,562,213 to Cabilly et al., and published PCT application WO 02/18901, both entitled “Electrophoresis Apparatus for Simultaneous Loading of Multiple Samples”; and published U.S. Patent Application 2002/0134680 A1, to Cabilly et al.; and published PCT application WO 02/071024, both entitled “Apparatus and Method for Electrophoresis”.

As discussed herein, a gel of the invention can be divided, in certain examples, into three functional zones: A, B and C. Zone A is an ion reservoir, adjacent to cathode. In one embodiment, the volumes of Zones A and C are each less than twice the volume of Zone B. In another embodiment, the volume of at least Zone A cr Zone C is less than twice the volume of Zone B. Zone B, which includes a running zone, is the area in which the molecule is separated and viewed. Zone C is the area between Zone B and anode, and is also an ion reservoir. In a preferred embodiment, Zone A has a volume of 4.5 ml, Zone B has a volume of 16.5 ml, and Zone C has a volume of 2.5 ml. In another preferred embodiment, Zone A has a volume of 2.5 ml, Zone B has a volume of 40 ml, and Zone C has a volume of 6 ml.

The ion reservoir may be in semi-solid form, in which the ion reservoir is incorporated within a porous substance such as a gel matrix. Thus, the “electrolyte solution” is present along the entire length of cassette, and includes both the running zone, Zone B, and the ion reservoir sources, Zones A and C.

In certain aspects of the invention a photoinitiator or photocatalyst is included only in zone B and zone A and C includes linear acrylamide.

In certain embodiments, provided herein is a system for electrophoresis, such as a high-throughput system, that includes:

(a) a gel according to the present invention; and

(b) a power supply comprising a power regulator.

In certain aspects, the power regulator provides constant power over a period of time sufficient for a set of proteins to resolve. The set of proteins, for example, can include at least two proteins having molecular weights selected from the group consisting of 20 kDa, 40 kDa, 60 kDa, 120 kDa and 220 kDa.

The power supply, in certain illustrative examples, is an E-Base™ power supply substantially as described herein. The system provided herein can include a series of interconnected bases, including a main base unit that plugs into an electrical outlet and a plurality of base units that are optionally included in the system. The plurality of additional base units can receive power through the main base unit or can otherwise be electrically connected to the main base unit.

In certain aspects, the system includes a gel assembly, itself a separate embodiment of the invention, that includes a composite agarose/acrylamide separation gel as disclosed herein, and a cassette. In certain examples, the gel assembly physically and electrically connects to a base unit such that an electrode within a separation gel is electrically connected through the cassette to the base unit or directly to the base unit. In one aspect of the present invention, the cassette is a substantially closed cassette that includes a three dimensional running area having a bottom wall and side walls and a top wall having a specified thickness. Cassette is substantially closed in that it is enclosed by walls, but it can also include vent holes and apertures.

The bottom wall and top wall of the cassette can be made of any suitable UV transparent material, such as the TPX plastic commercially available from MITSUI of Japan or the Polymethylmethacrylate (PMMA) plastic commercially available from Repsol Polivar S.P.A. of Rome, Italy. The cassette can include vent holes to allow for gaseous molecules that might be generated due to the electrochemical reaction (e.g., oxygen and/or hydrogen) to be released. In one embodiment, vent holes range in diameter from 0.5-2 mm. In a preferred embodiment, vent holes are 1 mm in diameter.

A plurality of wells can be introduced into a gel of the present invention, by using a “comb” having a row of protruding teeth positioned so that the teeth project into the gel layer while it sets. In one embodiment, the plurality of wells ranges from 1-200 wells. In another embodiment, the plurality of wells ranges from 8-12 wells. In another embodiment, the plurality of wells includes 96-104 wells. In another embodiment, the plurality of wells includes 48-56 wells. In one illustrative example of a gel of the invention, the gel further includes a comb. Furthermore, the gel can be included within a package such as a plastic pouch, for example to facilitate shipment to a customer. The package and/or the cassette can include a barcode.

When a gel has set after photoactivation as discussed herein, the comb is removed to leave a row of wells, or holes, in the layer. In one embodiment, wells are dimensions of 0.5-5 mm wide, 1-5 mm long, and 3-5 mm deep, and are used to introduce samples of the molecules to undergo molecular separation. One row or several rows may be formed.

As mentioned above, the cassette can also contain electrodes that when connected to an electric field, drive electrophoresis. The electrodes can be two conductive electrodes running along the width of the cassette. The system can also include a support, or base units, for connecting conductive elements of cassette to the power source. In one embodiment, the support configured to connect to one or more gels simultaneously. Further, the system optionally includes a camera for documentation, and a light source for visualization. In one embodiment, the light source is of variable wavelengths. In another embodiment, the light source is a UV light source. A colorimetric or chromogenic dye capable of interacting with molecules undergoing electrophoresis may be added so as to enable visualization while the molecules are in situ.

A typical method for staining electrophoretic media in a gel format that can be carried out at ambient temperature includes the steps of fixing the gel (e.g., incubating, the gel in an aqueous solution having about 40% ethanol and about 10% acetic acid for about 1 hour); rinsing the fixed gel one or more times with distilled water for about 10 minutes; incubating the gel in a staining solution for about 1 hour; and washing the gel one or more times with water or a buffer, such as one comprising sodium phosphate at a concentration of from about 5 to about 100 mM, e.g., 5, 10, 15, 20, 25, or 50 mM, the buffer having a pH of from about 6 to about 8, e.g., 7.0, 7.1, 7.2, 7.3, 7.4, 7.5, 7.6, 7.7, 7.8 or 7.9.

In another embodiment, the present invention provides an immunoblot transfer gel (1620) with sufficient porosity to retain sufficient blotting buffer to maintain a substantially or entirely uniform electrical field across a separation gel (1630) of the present invention having proteins being transferred to an immunoblot membrane (1640), and sufficient pliability to compensate for surface irregularities (1660) in a surface of a separation gel (1630) of the present invention in an immunoblot assembly (1600). When proteins are electrophoretically transferred from separation gels (1630) to transfer membranes (1640) (immunoblotting) using a semi-dry blotting apparatus, as discussed in further detail herein, the buffer used to support transfer is contained within pieces of absorbent paper (blotting paper) soaked in transfer buffer that are placed on either side of the separation gel to be transferred. This blotting ‘sandwich’ is then placed between electrode plates for transfer, usually with some amount of compression to maintain contact between sandwich components.

The immunoblot transfer gel or pad (1620) provided in one embodiment of the present invention is sufficiently porous to incorporate the transfer buffer within a solid but pliable gel- or pad matrix under the compression conditions in a blotting sandwich. The secondary function of the transfer gel or pad (1620), and the importance of pliability, is to conform to surface irregularities of the separation gel in an immunoblot assembly such that the irregularities do not manifest on a second face of the gel of the immunoblot assembly while being compressed within an immunoblot assembly during immunoblotting. In one aspect of the invention, the transfer gel (1620) further comprises an electrode imbedded therein. The electrode is placed in electrical communication with a power supply during immunoblotting to cause proteins to migrate from a separation gel to a protein binding transfer membrane.

In certain embodiment of the separation gels of the present invention, such as E-PAGE™ gels, irregularities that take the form of nubs or surface protrusions of gel matrix, created by the gel casting process, protrude above the gel plane when the gel is removed from its' cassette, as is required before transfer. These nubs, when compressed inside a semi-dry blotting device (such as the Bio-Rad Trans-Blot® SD or Major Science Semi Dry devices) can cause distorted protein transfer patters, either from blotting paper pressing back against the E-PAGE™ gel and the transfer membrane or by forming gaps between the E-PAGE™ gel and blotting paper on top of the gel nubs. By conforming to the separation gel, an immunoblot transfer gel (1620) provided herein allows compression of the E-PAGE™ gel and blotting sandwich components without distortion of blotting sandwich components and accompanying transfer distortions. Accordingly, a transfer gel (1600) of the present invention allows distortion-free immunoblotting of proteins from gels provided herein, such as E-PAGE™ gels. In certain aspects of the invention, the transfer gel comprises an electrode, imbedded therein.

The immunoblot transfer gel (1620), is approximately the same length and width as a separation gel used in an immunoblotting reaction. In one aspect, both the immunoblot transfer gel (1620) and a separation gel (180) are between 1 and 10 mm thick, for example, between 1 and 8 mm thick, 2 and 6 mm thick, or between 3 and 5 mm thick, or 3-4 mm thick. In one illustrative example, the separation gel is 4 mm thick and the transfer gel is 3-4 mm thick.

In certain aspects, the immunoblot transfer gel comprises immunoblotting transfer buffer, which can be used as the liquid component during manufacture of the immunoblot transfer gel (1620). Formulations of immunoblotting transfer buffers are known and can include, for example, a buffer, such as a Tris buffer, glycine, and a solvent such as methanol. As a non-limiting example, the transfer buffer can include 25 mM Tris—192 mM Glycine −15% methanol. In another non-limiting example, the transfer buffer is NuPAGE® Transfer Buffer (Invitrogen). The transfer buffer can also include an antioxidant.

Virtually any compound known to form a gel or a pad that can meet the requirements set out herein for the transfer gel (1620) can be used. In a preferred example, the gel is made of agarose, acrylamide, or combinations of agarose and acrylamide, or other matrix materials used for the preparation of electrophoresis gels could be used. In one illustrative example, the transfer gel is a 1-3% agarose gel. Other specific gel compositions can be determined by testing various gel formulations and assuring that the gel is sufficiently compliant to conform to the separation gel nubs yet sufficiently strong for easy handling.

In an illustrative embodiment, immunoassay transfer gels (1620) provided herein are prepared by dissolving a 1% agarose gel solution in Hispangar D-5 agarose with 1× NuPAGE® Transfer Buffer (Invitrogen, Carlsbad, Calif.), plus 1:1000 NuPAGE® (Invitrogen) Antioxidant as the liquid components. This solution is poured into a mold to cool and solidify. It is important that the mold have a flat bottom so that transfer gels of uniform thickness are made. After the agarose has cooled, the gel is trimmed to proper size and placed in 1× NuPAGE transfer buffer (plus antioxidant) to await use (this keeps the gel from drying out while the E-PAGE™ gel is being run). To use, the immunoblot transfer gel (1620) is placed on the cathode (1610) side of the E-PAGE™ gel (1630) during normal blot sandwich (1600) assembly, just as if it were a piece of blotting paper, as is normally used in semi-dry western blotting. The cathode side of an E-PAGE gel (1630), when it is properly positioned in an immunoblot (e.g. Western blot) sandwich, is the so called ‘well side’ of the gel—the side that the wells open toward and from which gel nubs (1660) protrude. The immunoblot transfer gel (1620) conforms to the gel protrusions (1660), preventing pressure from the cathode electrode (1610) and blot paper above from pushing the protrusions (1660) back against the blotting membrane (1640) or from allowing gaps between the E-PAGE gel (1630) and blot paper (1640) from forming. This allows an even electric field (1670) to be delivered to the proteins in the E-PAGE™ gel (1630), producing, non-distorted transfer.

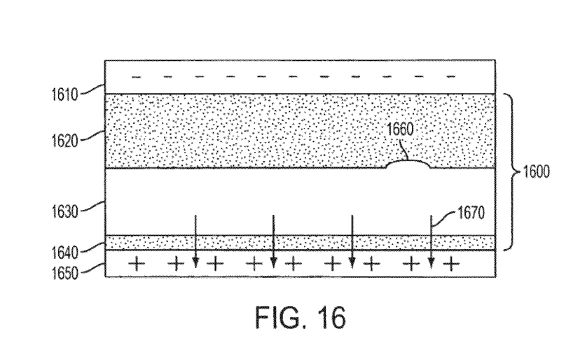

As illustrated in FIG. 16, in a related embodiment, provided herein is an immunoblot transfer assembly (1600), shown in a side view. The assembly comprises an immunoblot transfer gel or pad (1620) of the present invention, overlaying a separation gel (1630), which in turn overlies a transfer membrane (1640), such as a nitrocellulose or PDF membrane, the separation gel comprises a surface that is not entirely flat. For example, the separation gel may include bumps or nubs, for example in regions around the sample loading wells.

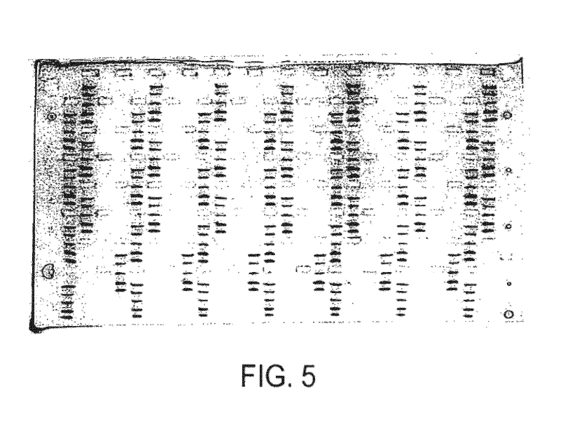

In certain aspects, as illustrated in FIG. 5, the transfer gel or pad (1620), preferably a gel, can overlay the separation gel (1630) on the cathode (1610) side of the separation gel. Overlaying or imbedded within the immunoblot transfer gel (1620) is a cathode (1610). Between the cathode (1610) and transfer gel (1620) in certain aspects of the invention, are one or more pieces of filter paper. Underlying the separation gel (1630) is a transfer membrane (1640) which overlies an anode (1650). In certain aspects, one or more pieces of filter paper are wedged in between the anode and the transfer membrane. The immunoblot assembly is typically held together by a blotting device.

In another aspect, the present invention provides a method for transferring one or more proteins from a separation gel to a transfer membrane. The method comprises providing an immunoblot transfer assembly (1600) comprising a transfer gel (1620) overlaying a separation gel (1630), which overlays a transfer membrane (1640) within a blotting device, and introducing an electric current through the immunoblot transfer assembly (1600) to force proteins located within a separation gel to contact a transfer membrane, thereby transferring the one or more proteins. The separation gel typically has surface irregularities which but for the presence of the transfer gel would cause protein band distortion during transfer. In certain aspects, the separation gel eliminates 75%, 80%, 85%, 90%, 95%, 99% or all of the protein band distortion. In one aspect, the separation gel is an acrylamide/agarose gel of the present invention. In another aspect, the transfer membrane and the separation gel are between 2 and 8 mm thick, for example 3-5 mm thick. The blotting, is typically carried out while the transfer assembly (1600) is in a horizontal orientation. The invention is particularly useful for horizontal semi-dry blotting. As a specific example, the method can be carried out as follows:

Immediately following an electrophoresis run, a separation gel is removed from the cassette and blotted. Blotting is carried out by laying the separation gel on a flat surface well side up in a tray. Remnant gel pieces are removed by gently rubbing a gloved finger over the well side of the separation gel. The separation gel will likely still have surface irregularities such as nubs (i.e. gel protrusions) near wells. An amount of 1× NuPAGE® Transfer Buffer (Invitrogen) sufficient to fill all the wells of the separation gel is poured over the gel. An immunoblot transfer gel pre-soaked in transfer buffer is laid on top of the gel, and any trapped air bubbles are removed by gently using a glass pipette as a squeegee across the surface of the transfer gel. Next a piece of pre-soaked filter paper is laid on top of the immunoblot transfer gel, and any trapped air bubbles were removed by gently using a glass pipette as a squeegee across the surface of the filter paper. The pre-soaked filter paper can be precut by a provider to match the length and width of the separation gel and/or the transfer gel. This assembly is turned over onto a clean flat surface so that the separation gel, transfer gel, and filter paper are facing downwards. A piece of pre-soaked transfer membrane (e.g., nitrocellulose) is placed on the side of the separation gel that is now on top. Another pre-soaked, and optionally pre-cut piece of filter paper is placed, on top of the membrane and air bubbles are removed as above. The assembly is positioned for electrophoretic transfer from the gel to the transfer membrane using an Invitrogen XCell II™ Blot Module and was run at 35 V for 1 hour. The transfer membrane is separated from the assembly and contacted with the primary antibody, at an effective dilution. Bound primary antibody is detected using the an anti-mouse antibody-conjugate.

In another embodiment, the present invention relates to pre-cut membranes for use in a western blotting procedure, wherein the sheets of membrane (e.g. nitrocellulose) are pre-cut to substantially match the dimensions as the pre-cast gel to facilitate blotting. Such pre-cut membranes may be supplied separately, or combined with pre-cast gels in kits for use in blotting. In other embodiments, pre-cut membranes made of materials other than nitrocellulose are used, such as Invitrolon™ (PVDF). Nitrocellulose and PVDF membranes having different pore sizes (e.g. 0.45 μm or 0.22 μm) may be used.

Filter papers for use in blotting may also be pre-cut to substantially match the dimensions of the pre-cast gels in some embodiments of the present invention. Filter papers of various thicknesses (e.g. 0.8 mm and 2.5 mm) may be used. Pre-cut filter papers may also be stacked to produce a greater thickness of paper (e.g. 6-8 mm). Filter papers may be stacked two, three, four, five, six, seven, eight, nine, ten or more layers thick. For example, a stack of filter papers 6-8 mm thick can be obtained using three 2.5 mm filter papers or eight 0.8 mm filter papers.

In other embodiments, pre-cut membranes and pre-cut filter papers are combined to form pre-cut membrane/filter paper sandwiches. Use of such pre-cut filter papers and membrane/filter paper sandwiches is described in detail at Example 5.

Gels, membranes, filter papers and membrane/filter paper sandwiches, being generally planar, have dimensions of length, width, and thickness. In some embodiments of the present invention, the length and width, but not the thickness, of the pre-cut membranes, filter papers, and membrane/filter paper sandwiches is selected to substantially match the length and width of the gel. A substantial match between the dimensions of pre-cut membranes, filter papers, and membrane/filter paper sandwiches and a gel does not require that the dimensions be the same. In some embodiments, membranes or filter papers substantially match the dimensions of a gel by extending only slightly beyond the edges of the gel, i.e. the membranes or filter papers are slightly larger than the gel in length, width, or both.

In some embodiments of the present invention, pre-cut membranes, pre-cut filter papers, and/or pre-cut membrane/filter paper sandwiches are included in kits. Such kits comprise one or more item selected from the group consisting of pre-cut membranes, pre-cut filter papers, and pre-cut membrane/filter paper sandwiches.

Another type of electrophoresis is isoelectric focusing (IEF) or electrofocusing. TEE, which can be carried out in an electrophoretic medium or in solution, involves passing a mixture through a separation medium which contains, or which may be made to contain, a pH gradient or other pH function. The device or gel has a relatively low pH at one end, while at the other end it has a higher pH. IEF is discussed in various texts such as Isoelectric Focusing by P. G. Righetti and J. W. Drysdale (North Holland Publ., Amsterdam, and American Elsevier Publ., New York, 1976).

The charge on a protein or other molecule depends on the pH of the ambient solution. At the isoelectric point (pI) for a certain molecule, the net charge on that molecule is zero. At a pH above its pI, the molecule has a negative charge, while at a pH below its pI the molecule has a positive charge. Each different molecule has a characteristic isoelectric point. When a mixture of molecules is electrophoresed in an IEF system, an anode (positively charged) is placed at the acidic end of the system, and a cathode (negatively charged) is placed at the basic (alkaline) end. Each molecule having a net positive charge under the acidic conditions near the anode will be driven away from the anode. As they electrophorese through the IEF system, molecules enter zones having less acidity, and their positive charges decrease. Each molecule will stop moving when it reaches its particular pI, since it no longer has any net charge at that particular pH. This effectively separates molecules that have different pI values. The isolated molecules of interest can be removed from the IEF device by various means, or they can be stained or otherwise characterized.

Some types of IEF systems generate pH gradients by means of “carrier ampholytes.” These are synthetic ampholytes that often have a significant amount of buffering capacity. When placed in an IEF device, each carrier ampholyte will seek its own isoelectric point. Because of their buffering capacity, many carrier ampholytes will establish a pH plateau rather than a single point. By using a proper mixture of carrier ampholytes, it is possible to generate a relatively smooth pH gradient for a limited period of time. Such mixtures are sold commercially under various trade names, such as Ampholine (sold by LKB-Produkter AB of Bromma, Sweden), Servalyt (sold by Serva Feinbiochemica of Heidelberg, FRG), and Pharmalyte (sold by Pharmacia Fine Chemicals AB, Uppsala, Sweden). The chemistry of ampholyte mixtures is discussed in various references, such as U.S. Pat. No. 3,485,736; Matsui et al., Methods Mol. Biol. 112:211-219 (1999); and Lopez, Methods Mol. Biol. 112:109-110 (1999).

In IEF in Immobilized pH gradients (IPG), ampholytic ions having multiple ionizable groups with differing pKa values such as proteins are forced to reach a steady-state position along pH inclines of various scopes and spans (see Righetti et al., Electrophoresis 15:1040-1043, 1994; Righetti et al., Methods Enzymol. 270:235-255, 1996; and 2-D Electrophoresis using immobilized pH gradients—Principles and Methods, Edition AC, Berkelman, T. and T. Stenstedt, Amersham Biosciences, Freiburg, Germany, 1998.). In one popular version of IPG, the pH gradient is in the form of a strip and is referred to as a “strip gel” or a “gel strip” that can be used in appropriate formats. See, by way of non-limiting example, published PCT patent applications WO 98/57161 A1, WO 02/09220 A1, published U.S. patent application US 2003/0015426 A1, and U.S. Pat. Nos. 6,599,410; 6,156,182; 6,113,766; and 6,495,017.

Two-dimensional (2D) electrophoresis techniques are also known and involve a first electrophoretic separation in a first dimension, followed by a second electrophoretic separation in a second, orthogonal dimension. In a common 2D electrophoretic method, proteins are subjected to IEF in a polyacrylamide gel in the first dimension, resulting in separation on the basis of isoelectric point (pI), and are then subjected to SDS-PAGE in the second dimension, resulting in further separation on the basis of size (O'Farrell, J. Biol. Chem. 250:4007-4021, 1975).

Electrophoresis also includes techniques known collectively as capillary electrophoresis (CE). Capillary electrophoresis (CE) achieves molecular separations on the same basis as conventional electrophoretic methods, but does so within the environment of a narrow capillary tube (25 to 50 μm). The main advantages of CE are that very small (nanoliter) volumes of sample are required; moreover, in a capillary format, separation and detection can be performed rapidly, thus greatly increasing sample throughput relative to gel electrophoresis. Some non-limiting examples of CE include capillary electrophoresis isoelectric focusing (CE-IEF) and capillary zone electrophoresis (CZE).

Capillary zone electrophoresis (CZE) is a technique that separates molecules on the basis of differences in mass to charge ratios, which permits rapid and efficient separations of charged substances (for a review, see Dolnik, Electrophoresis 18:2353-2361, 1997). In general, CZE involves introduction of a sample into a capillary tube, i.e., a tube having an internal diameter from about 5 to about 2000 microns, and the application of an electric field to the tube. The electric potential of the field both pulls the sample through the tube and separates it into its constituent parts. Each constituent of the sample has its own individual electrophoretic mobility; those having greater mobility travel through the capillary tube faster than those with slower mobility. As a result, the constituents of the sample are resolved into discrete zones in the capillary tube during their migration through the tube. An on-line detector can be used to continuously monitor the separation and provide data as to the various constituents based upon the discrete zones.

CZE can be generally separated into two categories based upon the contents of the capillary columns. In “gel” CZE, the capillary tube is filled with a suitable gel, e.g., polyacrylamide gel. Separation of the constituents in the sample is predicated in part by the size and charge of the constituents traveling through the gel matrix. This technique, sometimes referred at as capillary Gel Electrophoresis (CGE), is described by Hjerten (J. Chromatogr. 270:1, 1983), and is suitable for resolving macromolecules that differ in size but have a constant charge-to-mass ratio (Guttman et al., Anal. Chem. 62:137, 1990).

In “open” CZE, the capillary tube is filled with an electrically conductive buffer solution, and an electric potential is applied to the tube. The capillary wall becomes negatively charged when brought into contact with buffer at basic and neutral pH, but since the charges are fixed they are unable to migrate toward the anode. In contrast, positively charged ions in solution, e.g. H₃O⁺, countermigrate toward the cathode, resulting in a net migration of water toward the cathode during the run. This electroendosmotic flow provides a fixed velocity component which drives both neutral species and ionic species, regardless of charge, towards the cathode. Fused silica is principally utilized as the material for the capillary tube because it can withstand the relatively high voltage used in CZE, and because the inner walls of a fused silica capillary ionize to create the negative charge which causes the desired electroendosmotic flow. The inner wall of the capillaries used in CZE can be either coated or uncoated. The coatings used are varied and known to those in the art. Generally, such coatings are utilized in order to reduce adsorption of the charged constituent species to the charged inner wall. Similarly, uncoated columns can be used. In order to prevent such adsorption, the pH of the running buffer, or the components within the buffer, are manipulated.

IFE is a two-stage procedure utilizing agarose gel protein electrophoresis in the first stage and immunoprecipitation in the second stage. The specimen or sample is typically serum, urine, or cerebral spinal fluid. There are numerous applications for IFE in research, forensic medicine, genetic studies and clinical laboratory procedures and the greatest demand of IFE is in the clinical laboratory where it is primarily used for the detection and identification of monoclonal immunoglobin gammopathies.

In some embodiments, electrophoresis is carried out in formats suitable for high-throughput screening (HTS). Preferred HTS formats, as well as other formats for other electrophoretic applications, are described in:

U.S. Pat. No. 6,562,213 to Cabilly et al., and published PCT application WO 02/18901, both entitled “Electrophoresis Apparatus for Simultaneous Loading of Multiple Samples”;

U.S. Pat. No. 6,379,516 and published U.S. Patent Application 20020134680 A1, both to Cabilly et al., and published PCT application WO 02/071024, all entitled “Apparatus and Method for Electrophoresis”; and

U.S. Pat. Nos. 5,582,702; 5,865,974; and 6,379,516, all to Cabilly et al., and published PCT applications WO 96/34276 and WO 97/41070, all entitled “Apparatus and Method for Electrophoresis.”

Microfluidics involves the use of small compact devices to perform chemical and physical operations with minute volumes. For reviews, see Ehrlich et al., Trends Biotechnol. 17:315-319 (1999), and Stone et al., AIChE Journal 47:1250-1254 (2001), and references cited therein.

One aspect of microfluidics is the use of capillary electrokinesis to move materials in small volumes from one site to another on a solid substrate. Fluid samples move through tiny channels from one experimental site to another on the chip. The primary application for these devices is high-throughput screening, in which they are used to test biological samples more quickly at lower cost than conventional lab techniques. Preferably, numerous events may be simultaneously performed within a small area using orders of magnitude less reagent and sample than possible with conventional 96-well microtiter plates. Referred to commonly as “lab-on-a-chip”, these devices offer numerous advantages for performing chemical operations. For example, U.S. Pat. No. 6,054,277 to Furcht et al. discloses a genetic testing system that includes an integrated, unitary microchip-based detection device with microfluidic controls. The devices allow for mixing, carrying out chemical reactions, such as the polymerase chain reaction, genetic analysis, screening of physiological activity of drug candidates, and diagnostics, to mention only the more popular applications. The devices permit the use of much smaller amounts of reagents and sample, permit faster reactions, allow for easy transfer from one reaction vessel to another and separation of charged entities for rapid and accurate detection.

DNA chips are small flat surfaces on which strands of one-half of the DNA double-helix called DNA probes or oligonucleotides are bound. This type of chip can be used to identify the presence of particular genes in a biological sample. These chips, which contain hundreds or thousands of unique DNA probes, are also called DNA microarrays and can be manufactured using a variety of techniques, including semiconductor processing technology, on a variety of surfaces, including glass and plastic.

One application for biochips is the use of DNA microarrays for expression profiling. In expression profiling, the chip is used to examine messenger RNA (mRNA), which controls how different parts of the genes are turned on or off to create certain types of cells. If the gene is expressed one way, it may result in a normal muscle cell, for example. If it is expressed in another way, it may result in a tumor. By comparing these different expressions, researchers hope to discover ways to predict and perhaps prevent disease.

It will be readily apparent to one of ordinary skill in the relevant arts that other suitable modifications and adaptations to the methods and applications described herein may be made without departing from the scope of the invention or any embodiment thereof. The present invention will be more clearly understood by reference to the following Examples, which are included herewith for purposes of illustration only and are not intended to be limiting of the invention.

In another embodiment, provided herein is a kit that includes a separation gel according to the present invention. In illustrative examples, the separation gel is a pre-cast gel. In certain examples, the gel is a gel assembly that includes a gel and a cassette, which can be contained within a plastic package such as a plastic pouch. Furthermore, the gel can include a comb within the wells of the gel to retain the integrity of the wells. The comb can enter the gel through openings in the cassette at the wells. The kit can further include the following:

one or more sample loading buffers;

one or more protein standards;

one or more instruction sheets;

one or more pre-cut membranes for use in blotting, said pre-cut membranes having a length and a width, wherein the pre-cut membranes are pre-cut to match the length and width of the pre-cast gel; and/or

one or more immunoblot transfer gels for use in blotting, optionally having a length and a width that matches the length and width of the pre-cast gel. The gel can also include a gel cassette opener, such as a butterfly opener.

In another embodiment, the present invention provides a method for selling a separation gel and an immunoblot transfer gel, wherein a provider presents to a customer on a first wide area network screen or a first voice-driven menu, such as a menu on a phone system, a link to purchase the separation gel, such as a hyperlink on an Internet page, and presents to the customer on the first wide area network screen or first voice-driven menu, a link to purchase an immunoblot transfer gel, preferably of the same length and width as the separation gel. In one aspect, the first page also includes a link to purchase one or more sample loading buffers, one or more sample buffers for electrophoresis, one or more protein standards, or one or more pre-cut membranes for use in blotting, wherein the pre-cut membranes have a length and a width that matches the length and width of the pre-cast gel.

### Example 1

### Preparation of Gels

All chemicals used in the Examples were purchased from Sigma-Aldrich (Sigma-Aldrich Co., St. Louis, Mo.) unless indicated otherwise.

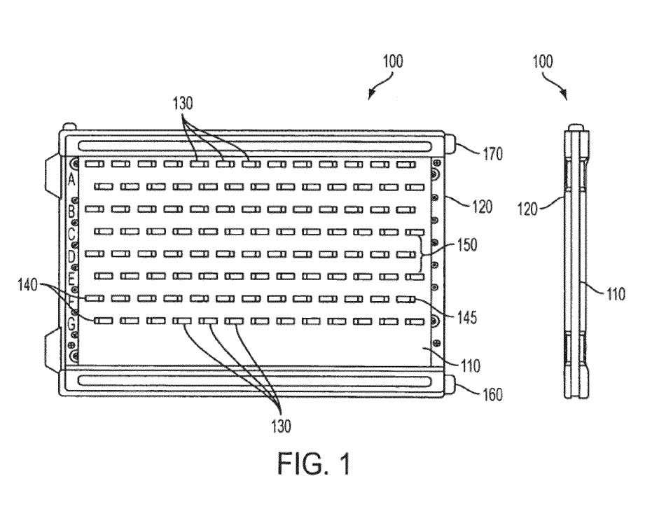

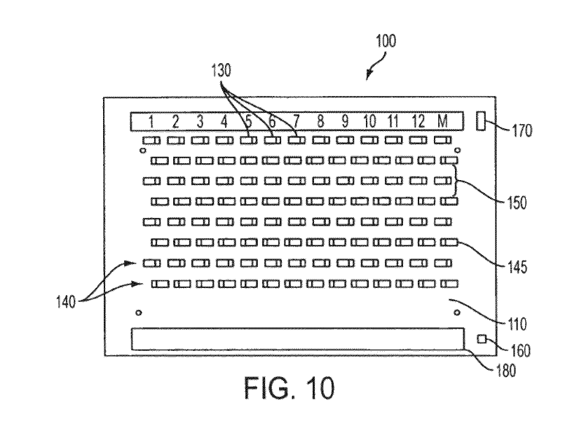

In an exemplary embodiment, gels of the invention were prepared in a “mini-gel” cassette having a staggered well format. See FIG. 1 and U.S. Pat. No. 6,562,213; published U.S. Patent Application 2002/0134680 A1; and published PCT applications WO 02/18901 and WO 02/071024. An exemplary gel of this format is referred to herein as an “E-PAGE™ 96 Gel”, which contains 96 sample lanes and 8 marker lanes and is compatible with standard 96-well plates, including but not limited to 96-well microtiter plates. The well spacing is designed to be compatible with multichannel pipettors and with 8-, 12- or 96-tip robotic loading devices. The protein separation range is from about 10 kilodaltons (kDa) to about 200 kDa in a separation distance of about 16 mm. An exemplary EPAGE™ 96 Gel assembly (100) is depicted in FIGS. 1 and 10. The specifications for assembly 100 are set forth in Table 1. Assembly 100 includes gel 110 and cassette 120. Gel 110 includes a plurality of loading wells 130 arranged in staggered rows, such as rows 140. Each well 130 is configured to be loaded with sample for electrophoretic analysis. During electrophoresis, the sample loaded in a given well 130 may be resolved in the associated sample lane 150. In the staggered row configuration depicted in FIG. 1, sample lane 150 can include the interstitial region between wells in the adjacent row. Each row 140 may also include additional well 145 for loading of electrophoretic marker standards.

TABLE 1

| | |
|---|---|
| Cassette (100) Size | 13.5 cm (L) × 10.8 cm (H) × |
| | 0.67 (W) cm |
| Gel (110) Thickness | 3.7 mm |
| Gel (110) Volume | 50 ml |
| Well (130, 145) | 3 mm |
| Depth | |
| Well (130, 145) | 3.8 mm × 1.8 mm |
| Opening | |
| Well (130, 145) | 3.3 mm × 1.1 mm |
| Bottom | |
| Running Distance | 16 mm |
| (150) | |
| Space Between Wells | 9 mm |

In another exemplary embodiment, gels of the invention are prepared in a “mini-gel” cassette having a 48-well format. A gel of this format is referred to herein as an “E-PAGE™ 48 Gel”, which contains 48 sample lanes and 4 marker lanes. Such 48-well gels may be preferable to 96-well gels in applications requiring increased resolution and/or applications with lower throughput requirements.

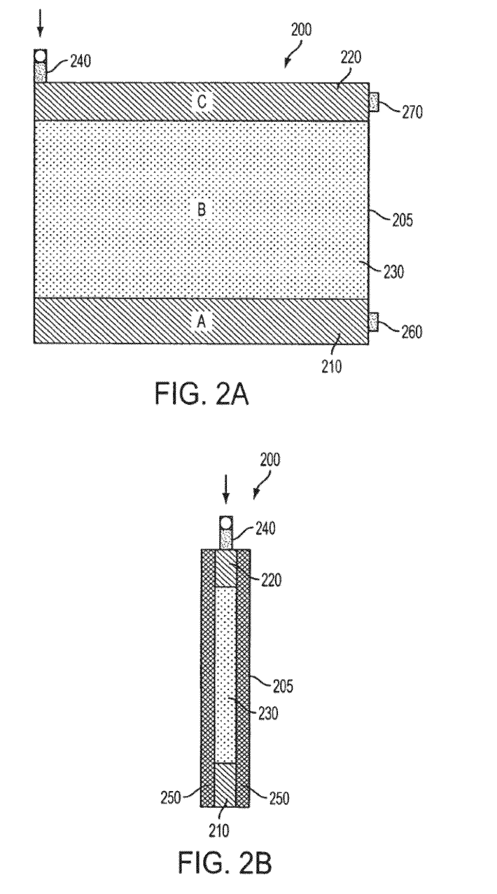

In the present invention, as illustrated in FIGS. 2A and 28, each cassette contains a gel made of three gel layers, labeled A, B and C, as depicted as cassette 200 with gel 205 in FIGS. 2A and 2B. First layer 210 and third layer 220, i.e., layers “A” and “C” have substantially the same composition, and are filled in the electrodes areas. Second layer 230, i.e., layer “B” is the running gel, i.e., the gel in which electrophoretic movement of macromolecules occurs.

For each gel cassette 200, 10 ml of first layer 210 and third layer 220 solution was prepared as follows: 0.209 g of Bis Tris (100 mM final concentration), 0.134 g of Tricine (75 mM final concentration), and 0.16 g of BES (75 mM final concentration) were dissolved in 9.55 ml of deionized water. Then 0.15 g (1.5% final concentration) of agarose D-5 (Hispanagar, S.A., Burgos, Spain), 0.4 ml (4% final concentration) glycerol and 0.047 ml (0.047% final concentration) of 10% SDS solution were added and the solution was boiled. After boiling, the solution was cooled to 80° C. and 0.1 g (1% final concentration) of linear polyacrylamide (MW 5,000,000 to 6,000,000; Polysciences Inc., Warrington, Pa.) was added and dissolved for 1 hour while stirring. After complete dissolution, 4 ml of this solution was passed through fill port 240 into, cassette 200 defined by parallel plates 250. The bold arrow in FIG. 2A indicates the direction of flow of material into the cassette 200 through fill port 240. The rest of the solution was kept at 80° C. for subsequent use. After 15 minutes at ambient temperature, first layer 210 of gel 205, which contacts anode 260, was completed.

Second layer 230 (“B”) solution was prepared as follows: A 50 ml solution was prepared for each cassette by dissolving 1.046 g (100 mM final concentration) of Bis Tris, 0.672 g (75 mM final concentration) of Tricine and 0.8 g (75 mM final concentration) of 2-[bis(2-hydroxyethyl)amino]ethanesulfonic acid (BES) in. 40.5 ml deionized water. To this was added 0.75 g (1.5% final concentration) of agarose (D-5, Hispanager), 2 ml (4% final concentration) of glycerol and 0.235 ml (0.047%) of a 10% SDS solution. The solution was then boiled. After boiling, the solution was cooled to 60° C., after which 7.5 ml of a 40% 19:1 prewarmed (60° C.) acrylamide-bisacrylamide solution (6% final concentration; AMRESCO, Inc., Solon, Ohio), 0.035 ml of Triethylamine (5 mM final concentration) and 0.025 ml of 0.1 M 1-hydroxycyclohexylphenyl ketone dissolved in propandiol (0.05 mM final concentration) was added. Forty-two (42) ml of this solution was passed through fill port 240 into cassette 200, and was allowed to solidify for 20 minutes at ambient temperature.

On top of second layer 230 (“B”), 4 ml of the third layer 220 (“C”) (identical to the solution used in first layer 210) was added to fill cassette 200 in cathode area 270. After solidifying (15 minutes at ambient temperature), fill port 240 was sealed.

For polymerization, gel-containing cassette 200 was exposed to 365 nm UV lamps for 20 minutes. After UV polymerization, pre-cast gel 205 was ready to use. After polymerization in an upright position, as illustrated in FIG. 2B, and before samples were loaded, the gel cassette holding the polymerized gels were laid down in a horizontal position, as illustrated in FIG. 2A. The gels can be stored for a prolonged period of time, for example days or months after polymerization and before use in electrophoresis. Therefore, the gels can be delivered to customers by a provider in a pre-cast format. Polypeptide samples, as discussed in certain Examples that follow, were loaded into wells, and during electrophoresis, polypeptides in the polypeptide sample were separated in gel B, as they migrated toward the anode. In one illustrative example, gels prepared in this fashion are run at 9 W constant power for 14 minutes.

### Example 2

### E-PAGE™ Protein Ladders and Loading Buffers E-PAGE™ MagicMark™ Protein Ladder

A protein standard that is particularly useful for use with the E-PAGE™ 96 Gel was developed from the MagicMark™ and MagicMark™ XP protein ladders (Invitrogen, Carlsbad, Calif.), and named the E-PAGE™ MagicMark™ protein ladder. Like other proteins, the MagicMark™ proteins can be stained with agents such as Coomassie Blue. In addition, however, the recombinant MagicMark™ proteins contain the immunoglobulin binding domain of protein G and can thus be directly bound and detected by most antibodies, irrespective of the antibody's antigenic specificity.

The original MagicMark™ protein ladder comprises nine proteins of known molecular weight, i.e., 20 kDa, 30 kDa, 40 kDa, 50 kDa, 60 kDa, 80 kDa, 100 kDa, 120 kDa; the MagicMark™ XP standard additionally contains a tenth protein of 220 kDa.

In contrast, the E-PAGE™ MagicMark™ protein ladder comprises five proteins having molecular weights of 20 kDa, 40 kDa, 60 kDa, 120 kDa and 220 kDa. That is, the E-PAGE™ MagicMark™ protein ladder is prepared essentially as are the MagicMark™ and MagicMark™ XP protein ladders, with the exception that protein standards having molecular weights of 30 kDa, 50 kDa, 80 kDa and 100 kDa are omitted from the formulation.

In one useful embodiment, the E-PAGE™ MagicMark™ Unstained Protein Standard is provided in a size of 250 μl, to be stored at −20° C.

The E-PAGE™ MagicMark™ Unstained Protein Standard is suitable for molecular weight estimation of proteins on E-PAGE™ pre-cast gels after staining or western blotting. The E-PAGE™ MagicMark™ Unstained. Protein Standard allows direct visualization of protein standard bands on a western blot without the need for protein modification or special detection reagents through the protein standard's IgG binding site.

Some important features of a preferred embodiment of the standard are: it consists of 5 recombinant protein bands in the range of 20-220 kDa; it is suitable for western blotting and molecular weight estimation; it is particularly designed for use on E-PAGE™ pre-cast gels; it can be visualized with alkaline'phosphatase-conjugated or peroxidase-conjugated antibody using chromogenic or chemiluminescent substrates; it can be visualized also with SimplyBlue™ SafeStain, silver stain, or fluorescent stains on E-PAGE™ pre-cast gels.

### Protein Ladder

In one embodiment, the standard can comprise 250 μl E-PAGE™ MagicMark™ Protein Standard in storage buffer comprising 125 mM Tris-HCl, pH 6.8; 10 mM DTT; 1% (w/v) SOS; 17.4% (w/v) glycerol; 0.025% Bromophenol Blue. The standard can be stored at −20° C. and is stable for at least 6 months at −20° C. To avoid repeated freezing and thawing, the standard can be aliquoted in small volumes and stored.

### Exemplary Directions for Use

In one embodiment, the E-PAGE™ MagicMark™ Unstained Protein Standard is supplied in a ready-to-use format. There is no need to heat or reduce the standard.

In an exemplary method, load 10 μl of the standard into the marker well of an E-PAGE™ 96 gel to obtain the best results, or load 5-10 μl for western blotting experiments. Perform electrophoresis as described in further detail herein below. It is recommended that different amounts of the standard be tested to determine the optimal amount of standard to use under your western blotting conditions. The amount of standard will depend on the sensitivity of your detection system, exposure time, and the binding affinity of MagicMark™ for your antibody species, as shown in Table 2.

TABLE 2
Affinity of E-PAGE ™ MagicMark ™ Proteins to Antibodies

| | Affinity of |
|---|---|
| Species | MagicMark ™ |
| Human, Horse, Cow | ++++ |
| Pig, Rabbit | +++ |
| Goat, Sheep, Hamster, | ++ |
| Guinea Pig, Rat, Mouse | |
| Chicken | + |

### Exemplary Directions for Blotting

After transferring proteins to a suitable membrane, perform the blocking step, primary antibody incubation step, and (if necessary) secondary antibody incubation step with the blot using a standard method of choice.

Visualize proteins using a chromogenic, chemiluminescent, or fluorescent detection system using the manufacturer's recommendations. After detection, you should observe 5 protein standard bands, as shown below.

### Exemplary Directions for Staining

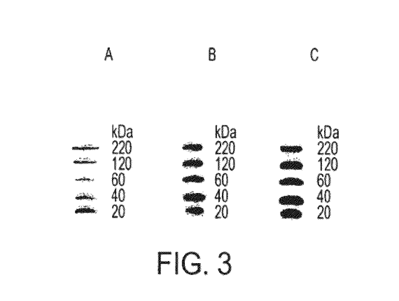

E-PAGE™ MagicMark™ Unstained Protein Standard (10 μl) was electrophoresed on a 6% E-PAGE™ 96 Gel and stained with SimplyBlue™ SafeStain (FIG. 3, lane A) as described herein below. In parallel, an aliquot of the standard (5 μl) was blotted onto a 0.45 μm nitrocellulose membrane, and detected with 1:5000 dilution of Anti-V5 Antibody from Invitrogen using the WesternBreeze® Anti-mouse Chemiluminescent Kit (FIG. 3 lane B) or WesternBreeze® Anti-mouse Chromogenic Kit (FIG. 3 lane C).

In a preferred embodiment, the E-PAGE™ MagicMark™ Standard is qualified on a 6% E-PAGE™ 96 Gel. After electrophoresis, the gel is stained with Coomassie® stain. The standard is also transferred onto a nitrocellulose membrane and detected with WesternBreeze® Anti-mouse Chromogenic or Chemiluminescent Kit (Invitrogen Corp., Carlsbad, Calif.). After staining and western detection, 5 standard bands must be detected for the product to pass.

Other suitable sets of protein standards can be prepared in like manner, as further detailed below.

### E-PAGE™ SeeBlue® Pre-Stained Protein Standard

As another example of protein ladders of the present invention that are useful in the gels of the present invention, a version of the SeeBlue® Pre-Stained Protein Standard (Invitrogen) particularly suitable for use on a gel of the present invention has been developed and is named E-PAGE™ SeeBlue® Pre-Stained Protein Standard.

E-PAGE™ SeeBlue® Pre-Stained Protein Standard is prepared essentially as are the original SeeBlue® Pre-Stained Protein Standard and SeeBlue® Plus2 Pre-Stained Protein Standard.

However, the original SeeBlue® Pre-Stained Protein Standard comprises proteins having approximate molecular weights on a NuPAGE® MES gel of 3 kDa (insulin, B chain), 6 kDa (aprotinin), 14 kDa (lysozyme), 18 kDa (myoglobin), 28 kDa (carbonic anhydrase), 38 kDa (alcohol dehydrogenase), 49 kDa (glutamic dehydrogenase), 62 kDa (BSA) and 188 kDa (myosin) and the SeeBlue® Plus2 Pre-Stained Protein Standard comprises proteins having approximate molecular weights on a NuPAGE® MES gel of 3 kDa (insulin B chain), 6 kDa (aprotinin), 14 kDa (lysozyme), 17 kDa (myoglobin red), 28 kDa (carbonic anhydrase), 38 kDa (alcohol dehydrogenase), 38 kDa (alcohol dehydrogenase), 49 kDa (glutamic dehydrogenase), 62 kDa (BSA), 98 kDa (phosphorylase B), and 188 kDa (myosin), whereas the molecular weights of the proteins in the E-PAGE™ SeeBlue® Pre-Stained Protein Standard, as measured on a 6% E-PAGE™ 96 gel, are 21 kDa, 42 kDa, 97 kDa, 173 kDa, and 261 kDa.

In one preferred embodiment, the standard is provided in a 500 μl size to be stored at 4° C.

The E-PAGE™ SeeBlue™ Pre-Stained Standard allows visualization of protein molecular weight ranges during electrophoresis and evaluation of western transfer efficiency. The E-PAGE™ SeeBlue® Pre-Stained Standard is particularly designed for use with E-PAGE™ pre-cast gels.

Features of a preferred embodiment of the standard include: it consists of 5 pre-stained protein bands (3 blue and 2 contrasting colors) in the range of 15-290 kDa; it is designed for particular use on E-PAGE™ pre-cast gels; it is supplied in a ready-to-use format.

In one embodiment, the standard comprises 500 μl of E-PAGE™ SeeBlue® Pre-Stained Standard stored in a buffer comprising Tris-HCl, formamide, and SDS. The standard is stored at 4° C. and is stable for 4 months at that temperature.

### Exemplary Directions for Use

The E-PAGE™ SeeBlue® Pre-Stained Standard is supplied in a ready-to-use format. There is no need to heat or add reducing agent.

Load 10 μl of the standard into the marker well of an E-PAGE™ 96 gel to obtain the best results, or load 5 μl for western blotting experiments.

In a preferred embodiment, the E-PAGE™ SeeBlue® Pre-Stained Standard shows 5 distinct bands when separated by electrophoresis on an E-PAGE™ 96 gel to pass.

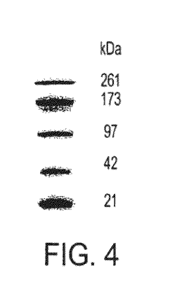

For example, the apparent molecular weights of protein bands in the E-PAGE™ SeeBlue® Pre-Stained Standard are shown in FIG. 4. A ten microliter aliquot of E-PAGE™ SeeBlue® Pre-Stained Standard was separated on a 6% E-PAGE™ 96 gel using procedures essentially as described herein below.

### Loading Buffers

A variety of loading buffers can be added to samples before they are transferred to the wells of a gel. Loading buffers of the invention include Buffer #1 and Buffer #2, which have the compositions disclosed in Table 3.

TABLE 3

| Name | CAS No. | Buffer #1 | Buffer #2 |
|---|---|---|---|
| Lauryl sulfate | | 8% | (none) |
| lithium salt | | | |
| Glycerol | 56-81-5 | 40% | 40% |
| Bromophenol blue | 62625-28-9 | 0.01% | 0.01% |
| Bis-Tris | 6976-37-0 | 4.1% | 4.1% |
| Tricine | 5704-04-1 | 5.3% | 5.3% |
| EDTA × 2H₂0 | 51898-34-1 | 0.1% | 0.1% |
| Water | | 42.49% | 50.49% |

These exemplary loading buffers are identical except for the presence of lauryl sulfate lithium salt (LDS), 8%, in Buffer #1. Buffer #2 is provided to dilute Buffer #1 in situations in which lower LDS concentrations are desirable including, by way of non-limiting example, in-gel staining procedures, such as those using SYPRO-Orange.

### Example 3

### Electrophoresis

The gel in FIG. 5 shows the results of electrophoresis of E-PAGE™ MagicMark™ protein ladder (Example 2) in a gel of the invention. Wells in the gel were loaded with 10 ;L of the E-PAGE™ MagicMark™ protein ladder and run at 9 W for 16 min in a staggered 96-well format. Following electrophoresis, the gel was removed from the cassette and stained with Coomassie Blue R-250 (Sigma) at a concentration of 0.02% and initially heated to about 50° C. for about 30 minutes. The stained protein bands were visible to the eye, and the image of the gel in FIG. 5 was obtained using a Hewlett Packard flat bed scanner with a transilluminator attachment. As can be seen, in each “lane” (one of which is boxed in the lower right corner of the gel), the E-PAGE™ MagicMark™ proteins were resolved based on their size (i.e., 220 kDa, 120 kDa, 60 kDa, 40 kDa and 20 kDa). The homogeneity of the electric field across the different lanes and rows is evident from the uniform distribution results of the standard markers.

### Example 4

### Western Blots

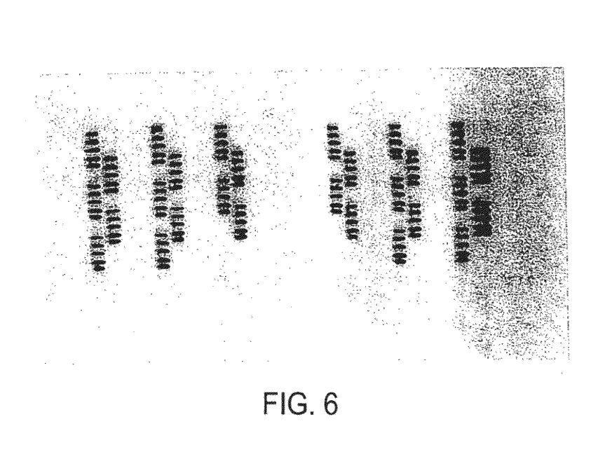

The gel in FIG. 6 is a Western blot of electrophoresed E-PAGE™ MagicMark™ protein standards (Example 2) in a gel of the invention. A 96-well staggered format gel was prepared and run essentially as is described in the preceding Examples, i.e., the electrophoresis was carried out at 9 W for 16 minutes, and the sample volume that was loaded was 5 ;L.

Immediately following the run, the gel was removed from the cassette and blotted. Blotting was carried out by laying the gel on a flat surface well side up in a tray. Remnant gel pieces were removed by gently rubbing a gloved finger over the well side of the gel. An amount of 1× NuPAGE® Transfer Buffer sufficient to fill all the wells of the gel was poured over the gel. A piece of pre-soaked filter paper was laid on top of the gel, and any trapped air bubbles were removed by gently using a glass pipette as a squeegee across the surface of the filter paper. This assembly was turned over onto a clean fiat surface so that the gel and filter paper were facing downwards. A piece of pre-soaked transfer membrane (nitrocellulose) was placed on the side of the gel that was now on top. Another pre-soaked piece of filter paper was placed on top of the membrane and air bubbles were removed as above. The assembly was positioned for electrophoretic transfer from the gel to the transfer membrane using an Invitrogen XCell II™ Blot Module and was run at 35 V for 1 hour. The transfer membrane was separated from the assembly and contacted with the primary antibody anti-V5, which is retained by the IgG-binding domains in the MagicMark™ proteins (see U.S. Pat. Nos. 5,082,773 and 5,108,894), at a 1:5000 dilution. Bound primary antibody was detected using the WesternBreeze® Anti-Mouse Chemiluminescent Kit (Invitrogen, Carlsbad Calif.). The image shown in FIG. 6 was captured using a Fuji LAS-1000 Luminometer (20 second exposure). As can be seen, in each “lane” (one of which is boxed in the lower right corner of the blot), the E-PAGE™ MagicMark™ proteins were resolved based on their size (i.e., 220 kDa, 120 kDa, 60 kDa, 40 kDa and 20 kDa) and specifically detected in the Western blot.

### Example 5

### Western Blotting with Pre-Cut Membranes and Filter Papers

Pre-cut filter papers, membranes and membrane/filter paper sandwiches may be used to facilitate western blotting experiments. Western blotting using pre-cut membranes, filter papers, or membrane/filter paper sandwiches may be performed using wet, semi-wet or semi-dry transfer procedures. In the semi-dry procedure described in this Example, MagicMark™ Standard, NuPAGE® Transfer Buffer, NuPAGE® antioxidant, E-PAGE™ gels, pre-cut E-PAGE™ membrane/filter paper sandwiches and pre-cut E-PAGE™ filter papers are obtained from Invitrogen (Carlsbad, Calif., USA).

Several wells of a 96-well 6% E-PAGE™ gel are loaded with 5 μl of MagicMark™ Standard. The gel is run according to the manufacturer's instructions, the gel cassette is opened and the anode and cathode sections of the gel are trimmed off using a gel knife or similar tool. Any pieces of gel on the surface are removed by rubbing the gel surface gently with a gloved finger and rinsing briefly with deionized water. The gel is incubated with shaking for 30 minutes in 200 ml. 2× NuPAGE® Transfer Buffer containing 1:1000 NuPAGE® antioxidant. If a 12% E-PAGE™ gel is used, rather than 6%, and if high molecular weight proteins are of interest, incubation is performed for 60 minutes rather than 30 minutes. In some embodiments, transfer buffer does not contain alcohol.

Proteins are then transferred to a membrane, and the proteins are detected, as follows. A semi-dry blotting procedure is employed. A pre-cut E-PAGE™ membrane/filter paper sandwich is used. The pre-cut E-PAGE™ membrane/filter paper sandwich comprises a stack of eight pieces of 0.8 mm filter paper (8.6 cm×13.5 cm) and a 0.45 μm nitrocellulose membrane (also 8.6 cm×13.5 cm). An additional eight pieces of 0.8 mm filter paper (8.6 cm×13.5 cm) are used in the transfer.

The membrane/filter paper sandwich and the eight additional filter papers are soaked in 2× NuPAGE® Transfer Buffer containing 1:1000 NuPAGE® antioxidant. If a PVDF membrane is used rather than nitrocellulose, the membrane must first be wetted in alcohol (methanol, ethanol or isopropyl alcohol) and rinsed with deionized water prior to soaking in transfer buffer. The membrane/filter paper sandwich is placed on the anode plate of the western transfer apparatus with the filter paper layers facing the anode plate. Air bubbles are gently rolled out using a roller, pipet or other suitable implement.

The gel is then placed on top of the upper surface of the membrane/filter paper sandwich, i.e. against the nitrocellulose membrane. The flat side of the gel is placed in contact with the nitrocellulose membrane, and the side with the wells is left facing upwards. Any trapped air bubbles are gently removed using a roller, pipet or other suitable implement. Gel wells are filled with 2× NuPAGE® Transfer Buffer containing 1:1000 NuPAGE® antioxidant, and the eight remaining pieces of filter paper are stacked on top of the gel. Any air bubbles are gently rolled out as described above. The cathode plate is then carefully placed on top of the stack and secured according to the blotting apparatus manufacturer's instructions. Care is taken during assembly to avoid disturbance of the stack of filter papers, membrane and gel.

Transfer is performed at 25 V (approximately 14 V/cm) for 60 minutes. If a 12% E-PAGE™ gel is used, rather than 6%, transfer is performed at 35 V (approximately 19.4 V/cm) for 60 minutes.

The nitrocellulose membrane is then exposed to a 1:1000 dilution of anti-His (C-term) mouse monoclonal antibody and developed with the WesternBreeze® Anti-mouse Chemiluminescent Kit (Invitrogen, Carlsbad, Calif., USA). The image is captured using a Fuji LAS-1000 Luminometer with a 30 second exposure.

### Example 6

### Other Photoinitiators

Gels and compositions of the invention are prepared using other photoinitiators. For example, benzophenone tetracarboxylic dianhydride can be used. To prepare a gel using this photoinitiator, the procedure used was essentially the same as described in the other Examples, with the following exceptions. The final TEA concentration was 10 mM and the 1-hydroxy-cyclohexyl-phenyl-ketone component was replaced with 0.125 ml of a stock solution of benzophenone tetracarboxylic dianhydride (Fluka, Buchs SG, Switzerland) to a final concentration of 25 ;M. The stock solution comprised 10 mM of benzophenone tetracarboxylic dianhydride in 50 mM Bis Tris and 50% propandiol.

### Example 7

### Power Supply and Instructions

In some embodiments, it is preferable to perform electrophoresis using a power supply that provides for constant power (measured in watts) during the electrophoretic run. That is, a preferred power supply includes a power regulator. Non-limiting examples of subsystems for a power regulator include a voltage regulator and a current regulator. Other optional features of a power supply are means to program the power supply to provide a set amount of power, voltage and/or current over a pre-set period of time.

A set of instructions that describe E-Base™ power supplies that are preferably used with certain gels of the invention (E-PAGE™ 96 Gel) follows.

The E-Base™ is an easy-to-use, pre-programmable, automated device designed to simplify electrophoresis of pre-cast E-PAGE™ 96, E-Gel® 48, and E-Gel™ 96 gels from Invitrogen. The E-Base™ is a base and a power supply combined in one device.

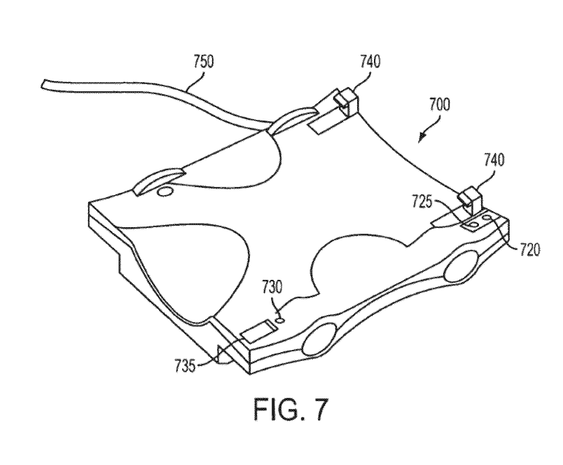

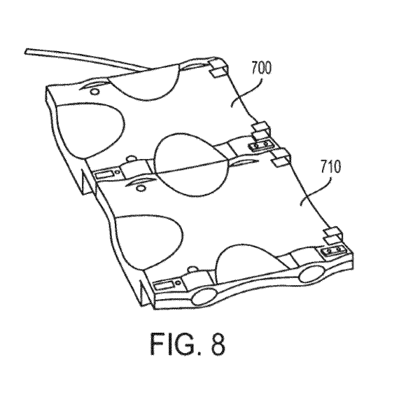

Two types of bases are available from Invitrogen: (1) The Mother E-Base™ (700) (Catalog no. EB-M03) has an electrical plug that can be connected directly to an electrical outlet and is used for electrophoresis of one E-PAGE™ 96, E-Gel® 48, or E-Gel® 96 gels available from Invitrogen; (2) The Daughter E-Base™ (710) (Catalog no. EB-D03) connects to the Mother E-Base™ (700), and together they can be used for the electrophoresis of two or more E-PAGE™ 96, E-Gel® 48, or E-Gel® 96 gels available from Invitrogen. The Daughter E-Base™ (710) does not have an electrical plug and cannot be used without a Mother E-Base™ (700). Mother and Daughter E-Base™ (710) units are shown in FIGS. 7 and 8.

The Mother E-Base™ (700) and Daughter E-Base™ (710) are 14.6 cm×15 cm×5.3 cm. The Mother E-Base™ (700) is 370 g and the Daughter E-Base™ (710) is 271 g. Both Mother and Daughter E-Base™ (710) Daughter have double insulation for safety, and are designed to operate at ambient temperatures from 5° C. to 40° C. Built-in Features include a digital timer display (00-99 minutes) (735), alarm, and light LED (730).

The SBS (Society for Biomolecular Screening) standard 96-well plate format of the E-Base™ fits on most robotic platforms allowing the loading and electrophoresis of gels on the E-Base directly on the robot.

### Mother E-Base™ (700)

Each Mother E-Base™ (700) has a pwr/prg (power/program) button (right side) (720) and a timer button (left side) (725) on the lower right side of the base (700). The lower left side of each Mother E-Base™ (700) contains a light LED (730) and a digital timer display (00-99) (735). The gel cassette (100) is inserted into the two electrode connections (740). The Mother E-Base™ (700) is connected to an electrical outlet with the electrical plug (750).

The E-Base™ is pre-programmed with two programs specific for each gel type: an EG Program for E-Gel® 96 gels, with a 12 minute run parameter; and an EP Program for E-PAGE™ 96 gels, with a 14 minute run parameter.

### Daughter E-Base™ (710)

The Daughter E-Base™ (710) is similar to the Mother E-Base™ (700) except the Daughter E-Base™ (710) does not have an electrical cord and cannot be connected to an electrical outlet. The Daughter E-Base™ (710) is connected to a Mother E-Base™ (700) or to another Daughter E-Base™ (710) (already connected to a Mother E-Base™ (700)). Once connected to a Mother E-Base™ (700), each Daughter E-Base™ (710) is designed to function independently of the Mother E-Base™ (700) or other Daughter E-Base™.

### Instructions for Use

You will need to select an appropriate program on the E-Base™ prior to inserting a gel into the E-Base™ as follows. Plug the Mother E-Base™ (700) into an electrical outlet using the electrical plug on the base. If using a Daughter E-Base™ (710), connect the Daughter E-Base™ (710) to a Mother E-Base™ (700) or another Daughter E-Base™ (710) connected to a Mother E-Base™ (700).

The display (735) will show EP or last program used (EP or EG) with no gel cassette.

Select the appropriate program based on the gel by pressing and releasing the pwr/prg (power/program) button (720). For an E-Gel® 96 gel, select program EG. For an E-Gel® 48 gel, select program EG and then manually change time to 20 minutes by pressing and holding the timer button (725) until 20 minutes is displayed (see below for details). For an E-PAGE™ 96 gel, select program EP.

### Setting the Timer

The initial default timer setting on an E-Base™ for program EG is 12 minutes and EP is 14 minutes. Follow instructions below to increase or decrease the time setting, if desired.

Do not run an E-Gel® 96 gel for more than 20 minutes, an E-Gel® 48 gel for more than 30 Minutes, and an E-PAGE™ 96 gel for more than 20 minutes.

To increase or decrease the default run time when no cassette is inserted on the base, use the following procedure.

Connect the Mother E-Base™ (700) to an electrical outlet. If you are using a Daughter E-Base™ (710), connect the Daughter E-Base™ (710) to the Mother E-Base™ (700) and then connect the Mother E-Base™ (700) to an electrical outlet.

Press and release the timer button (725) located on the lower right corner of the base to view the timer setting.

Press and hold the timer button (725) to increase the time continuously.

When you reach the desired default time, release the timer button (725).

If the timer button (725) is not released, the timer setting (735) will increase until it reaches 00. To begin cycling through the numbers again, starting from 00, press the timer button (725) again.

To increase the run time when a cassette (100) is inserted, press and release the timer button (725) to increase the time setting by 1-minute intervals or press and hold the timer button (725) to increase the time continuously.

To increase the run time while a run is in progress, see below. To manually interrupt or stop a run, see below.

### Running the Gel

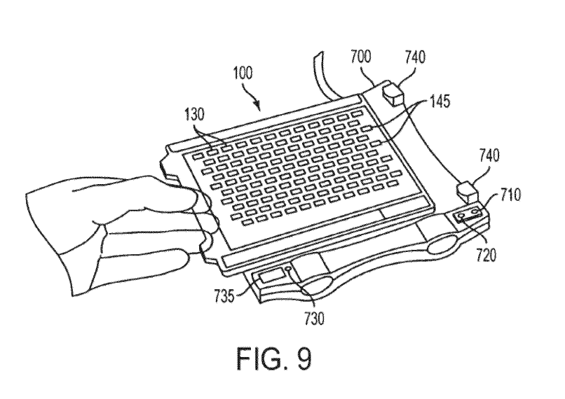

Open the package and remove the gel (100). Remove the plastic comb from the gel. Slide the gel into the two electrode connections (740) on the Mother E-Base™ (700) or Daughter E-Base™ (710) (see FIG. 9). The two copper electrodes (160, 170) on the right side of the gel cassette (100) must be in contact with the two electrode connections (740) on the base (700), as shown in FIG. 9.

When the gel is properly inserted into the base, a fan in the base will begin to run and a red light (730) will illuminate at the lower left corner of the base. The digital display (735) will show the appropriate time for a selected program or last time setting (Ready Mode).

Load the appropriate amount of DNA or protein samples into sample wells (130). Load water or sample buffer containing the same salt concentration as the sample into any remaining empty wells as described in the manual for each gel type.

Load DNA or protein markers in marker wells (145).

To begin electrophoresis, press and release the pwr/prg button (720) located on the lower right corner of the Mother E-Base™ (700) or Daughter E-Base™ (710).

The red light (730) will change to a green light and the digital display (735) will show the count down time while the run is in progress.

To add to the run time while the run is in progress, press the timer button (725) to select the desired time and then release the timer button (725).

To interrupt or stop a run in progress, see below.

The Mother E-Base™ (700) or Daughter E-Base™ (710) will signal the end of the run with a flashing red light (730) and rapid beeping for two minutes followed by a single beep every minute. At the end of the run, the digital display (735) will show the original time setting (not any time change that was made during the electrophoresis). The digital display (735) will also show the elapsed time (up to 19 minutes with a negative sign) since the end of the run.

Press and release the pwr/prg button (720) to stop the beeping. The light will turn to a steady red and the digital display (735) will show the last time setting.

Remove the gel cassette (100) from the Mother E-Base™ (700) or Daughter E-Base™ (710). You are now ready to capture an image of the gel.

Note: The bands in the gel will diffuse within 20-40 minutes.

The Mother E-Base™ (700) or Daughter E-Base™ (710) will signal the end of the run with a flashing red light (730) and rapid beeping for 2 minutes followed by a single beep every minute.

At the end of the run, the digital display (735) will show the original time setting (not any time change that was made during the electrophoresis). The digital display (735) will also show the elapsed time (up to 19 minutes with a negative sign) since the end of the run.

Press and release the pwr/prg button (720) to stop the beeping. The light (730) will turn to a steady red and the digital display (735) will show the last time setting.

Remove the gel cassette (100) from the Mother E-Base™ (700) or Daughter E-Base™ (710). You are now ready to capture an image of the gel. Note that the bands in the gel will diffuse within 20-40 minutes.

It is advisable to disconnect the Mother E-Base™ (700) from the electrical outlet when not in use for a prolonged period of time.

### Interrupting an Electrophoresis Run

You can interrupt an electrophoresis run at any time by pressing and releasing the pwr/prg button (720) to stop the current. The stopped current is indicated by a steady red light (730) and the digital display (735) will flash to indicate that the run was interrupted.

You can remove the gel (100) from the mother (700) or daughter base (710) to check the progress of the run. To continue the run from the point at which it was stopped, reinsert the gel and press and release the pwr/prg button (720). The light (730) changes to steady green and the digital display (735) shows the count down time.

To cancel the rest of the interrupted run, press and hold the pwr/prg button (720) for a few seconds. The digital display (735) will reset and the base will return to Ready Mode. If desired, you can then program a new run time as described on page 8 and rerun the gel.

In case of an external power failure (loss of electricity or the electrical cord is accidentally removed from the outlet), the run will continue when the power resumes. The Mother E-Base™ (700) or Daughter E-Base™ (710) will signal the end of the run as described on the previous page, except the light (730) will be an alternating red/green to indicate that an external power failure has occurred during the run.

### Maintaining E-Base™

The surfaces of the Mother E-Base™ (700) and Daughter E-Base™ (710) should be kept free of contaminants. To clean, disconnect bases from power source and wipe clean with a dry cloth. Do not attempt to open the Mother E-Base™ (700) or Daughter E-Base™ (710).

### E-Base™ Quick Reference Guide

A quick reference guide for operating the Mother E-Base™ (700) and Daughter E-Base™ (710) is provided at Table 4.

TABLE 4

| | | | | Digital |
|---|---|---|---|---|
| | | | Light | Display |
| Mode | Action | Sound | (730) | (735) |
| Base | Mother E- | 1 beep | No light | Without gel |
| plugged | Base ™ | | if a | cassette - |
| in | (700) | | cassette | EP, last |
| | connected | | is not | program |
| | to an | | inserted, | used (EP or |
| | electrical | | or red | EG) |
| | outlet | | light if a | With gel |
| | | | cassette | cassette in - |
| | | | is | last time |
| | | | inserted | setting |
| Ready | Gel | — | Steady | Default |
| (with no | cassette | | red | time |
| current | (100) | | | setting |
| flowing | inserted | | | (12 minutes |
| through | into a | | | for EG, 14 |
| gel) | base | | | minutes for |
| | | | | EP, or last |
| | | | | time |
| | | | | setting) |
| Run | Press and | — | Steady | Count down |
| | release | | green | time |
| | the | | | |
| | pwr/prg | | | |
| | button | | | |
| End of | Automatic | Continuous | Flashing | Negative |
| run | | beeping | red until | time |
| | | for | the timer | display |
| | | 2 minutes | button is | (00 to −19 |
| | | followed | pressed | minutes) |
| | | by a | | |
| | | single | | |
| | | beep every | | |
| | | minute | | |
| Run ends | Automatic | Continuous | Alternating | Negative |
| after an | | beeping | red | time |
| external | | for | and green | display (00 |
| power | | 2 minutes | | to |
| failure | | followed | | −19 |
| during | | by a | | minutes) |
| the run | | single | | |
| | | beep every | | |
| | | minute | | |
| Pause | Press and | — | With gel | Flashing |
| (manually | release | | cassette | time |
| end the | the | | in - | display |
| run) | pwr/prg | | steady | |
| | button | | red | |
| | (720) | | Without | |
| | during | | gel | |
| | the run | | cassette - | |
| | | | no | |
| | | | light | |
| Return to | Press and | — | Steady | Last time |
| Ready | release | | red | setting |
| mode | the | | | |
| after an | pwr/prg | | | |
| automatic | button | | | |
| stop | (720) | | | |
| Restart | Press and | — | Steady | Count down |
| after a | release | | green | time |
| manual | the | | | |
| stop | pwr/prg | | | |
| | button | | | |
| | (720) | | | |
| Return to | Press and | — | With gel | With gel |
| Ready | hold the | | cassette | cassette in - |
| mode | pwr/prg | | in - | last time |
| after a | button | | steady | setting |
| manual | (720) | | red | Without gel |
| stop | | | Without | cassette - |
| | | | gel | last |
| | | | cassette - | program |
| | | | no | setting |
| | | | light | |
| Failure | Press and | Continuous | | Flashing |
| | hold | loud | | “ER” |
| | pwr/prg | beeping | | |
| | button | | | |
| | (720) for | | | |
| | 2 seconds | | | |
| | and | | | |
| | remove | | | |
| | gel from | | | |
| | the base | | | |
| No | — | — | — | EP, last |
| cassette | | | | program |
| | | | | used (EP or |
| | | | | EG) |
| Timer | With gel | — | With gel | Time |
| setting | cassette | | cassette - | increases |
| | in - | | steady | by 1 minute |
| | Press and | | red | increments |
| | release | | | |
| | the timer | | | |
| | button | | | |
| | (725) | | | |
| | With and | — | With gel | Time |
| | without | | cassette | increases |
| | gel | | in - | continuously |
| | cassette - | | steady | and |
| | Press | | red | automatically |
| | and hold | | Without | stops at |
| | the timer | | gel | 00 |
| | button | | cassette - | |
| | (725) | | no | |
| | | | light | |
| Program | Press and | 1 beep | No light | Selected |
| setting | release | | | program EP |
| | the | | | or EG |
| | pwr/prg | | | |
| | button | | | |
| | when no | | | |
| | cassette | | | |
| | is | | | |
| | inserted | | | |
| | into the | | | |
| | E-Base ™ to | | | |
| | select | | | |
| | the | | | |
| | desired | | | |
| | program | | | |

Table 5 provides some solutions to the problems you might encounter during electrophoresis using E-Base™.

| Problem | Reason | Solution |
|---|---|---|
| No | Daughter E-Base ™ | Do not use the Daughter |
| current | (710) used without | E-Base ™ (710) without a |
| | a Mother E-Base ™ | Mother E-Base ™ (700). The |
| | (700) | Daughter E-Base ™ (710) |
| | | does not have an |
| | | electrical plug to |
| | | connect to an electrical |
| | | outlet. |
| | Copper contacts | Make sure that the copper |
| | (740) in the | contact in the base is |
| | Mother E-Base ™ | intact. |
| | (700) or Daughter | |
| | E-Base ™ (710) are | |
| | damaged due to | |
| | improper use | |
| | Expired or | Use fresh gel cassette. |
| | defective gel | Use properly stored gels |
| | cassette used | before the specified |
| | | expiration date. |
| | Gel cassette is | Remove cassette and |
| | not correctly | reinsert; a steady red |
| | inserted into the | light will be illuminated |
| | base | on the base when the |
| | | cassette is correctly |
| | | inserted and power is on. |
| Over-run | Accidentally | Select EG if you are |
| the gel | selected an | using E-Gel ® 48 or E-Gel ® |
| or need | incorrect program | 96 gels and EP if you are |
| more time | | using E-PAGE ™ 96 gels. |
| to run | | If you are at the |
| gel | | beginning of the run, |
| | | stop the run and select |
| | | the desired program. |
| | | If you are well into the |
| | | run, check the gel to see |
| | | where the loading dye is |
| | | running. Estimate the |
| | | amount of time remaining |
| | | and then manually stop |
| | | the run. |
| Failure | Defective cassette | Disconnect E-Base ™ and |
| Mode | | remove the gel cassette |
| indicated | | from the base. |
| by | | Press and hold the |
| flashing | | pwr/prg button (720) for |
| “ER”, and | | 2 seconds to return to |
| continuous | | Ready Mode. Use a fresh |
| loud | | gel cassette. |
| beeping | Cold cassette | Use a room temperature |
| | | cassette stored at room |
| | | temperature. Avoid |
| | | storing gel cassettes at |
| | | 4° C. |
| | Improper operating | Use E-Base ™ at room |
| | conditions | temperature |
| | | (20° C. to 25° C.) |

Operation of the E-Base™ is subject to the following conditions: indoor use; altitude below 2,000 meters; temperature between 5° and 40° C.; maximum relative humidity of 80%; installation categories (over voltage categories) II; pollution degree 2; mains supply voltage fluctuations not to exceed 10% of the nominal voltage (100-240V, 50/60 Hz, 500 mA); mains plug is a disconnect device and must be easily accessible.

The Mother C-Base™ (700) has been tested with up to three Daughter E-Bases™ (710) connected at one time. Do not attempt to open the Mother E-Base™ (700) or Daughter E-Base™ (710).

Additional products available separately from Invitrogen are listed in Table 6.

TABLE 6

| Product | Quantity | Catalog no. |
|---|---|---|
| E-Gel ® 96 1% Gels | 8 gels | G7008-01 |
| E-Gel ® 96 2% Gels | 8 gels | G7008-02 |
| E-Gel ® 48 4% Gels | 8 gels | G8008-04 |
| E-PAGE ™ 96 6% Gels | 1 kit | EP096-06 |
| E-Holder ™ Platform | 2 | EH-03 |

### Example 8

### Kit Instructions

Exemplary instructions for a kit or pre-cast gel, and associated equipment and solutions of the invention, are set forth in this Example. For optimal results, load each E-PAGE™ 96 Gel (100) within 30 minutes of removing the gel from the plastic pouch and run within 15 minutes of loading.

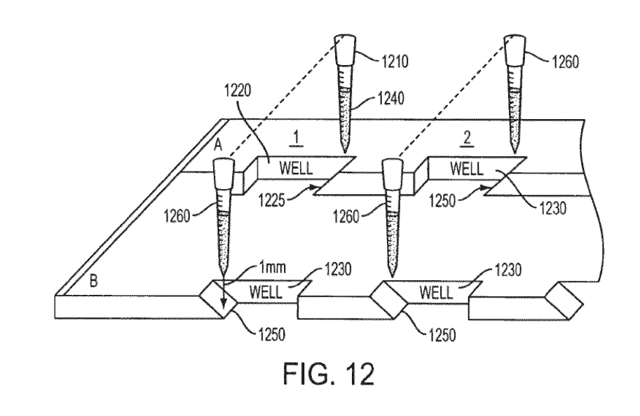

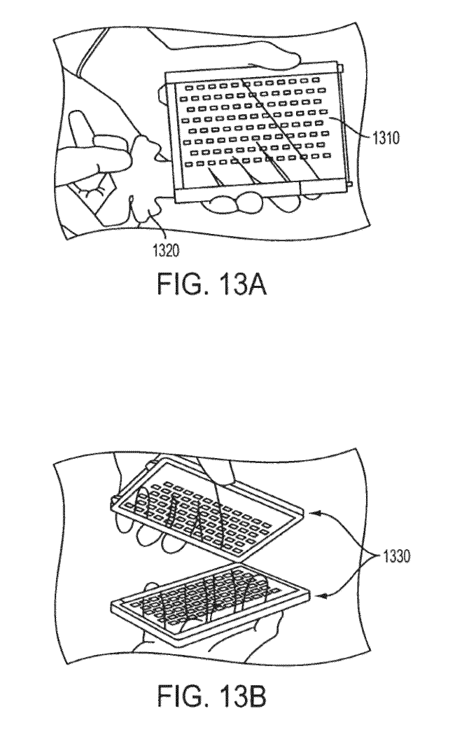

TABLE 7

| Step | Action |
|---|---|
| Prepare | Use up to 20 μg protein per lane of the E- |
| Sample | PAGE ™ 96 Gel (100). See page 5 for sample |
| | preparation. |
| Align | If you are using automated robotic loading, |
| Robotic | you need to align the robotic tip assembly |
| Tip | as described below. |
| Assembly | 1. Set the position of the first tip |
| (FIG. 12) | (1210) approximately 1 mm above the slope |
| | of the A1 well (1220) to ensure that the |
| | remaining tips are aligned above the slopes |
| | (1250) of the other wells (1230). |
| | 2. Refer to the manufacturer's manual |
| | for your robot to program this setting. |
| | Proceed to loading the gel. |
| Select | 1. Plug the Mother E-Base ™ (700) into an |
| Program | electrical outlet. |
| and Load | 2. If using a Daughter E-Base ™ (710), |
| | connect the Daughter E-Base ™ (710) to a |
| | Mother E-Base ™ (700) or another Daughter E- |
| | Base ™ (710) connected to a Mother E-Base ™ |
| | (700). |
| | 3. Select the program EP for E-PAGE ™ 96 |
| | Gels by pressing and releasing the pwr/prg |
| | (power/program) (720) button. The digital |
| | display (735) will show EP. |
| | 4. Remove gel from the package and |
| | remove the plastic comb from the gel. |
| | 5. Slide the gel into the two electrode |
| | connections (740) on the |
| | Mother E-Base ™ (700) or Daughter E-Base ™ |
| | (710). |
| | 6. Load samples into the gels using a |
| | multichannel pipettor or an automated |
| | liquid handling system. First load |
| | deionized water into each well (130) and |
| | then load your samples or protein molecular |
| | weight standard. If loading 5-10 μl of |
| | sample in loading buffer, first load 20 μl |
| | of deionized water. If loading 11-20 μl of |
| | sample in loading buffer, first load 19-10 μl |
| | deionized water. |
| | 7. Load the appropriate protein |
| | molecular weight marker in the marker wells |
| | (145) of the gel. |
| Electrophoresis | 1. Press and release the pwr/prg button |
| with | (720) located on the lower right corner of |
| E-Base ™ | the base to begin electrophoresis. |
| | 2. The Mother E-Base ™ (700) and Daughter |
| | E-Base ™ (710) will signal the end of the |
| | run with a flashing red light (730) and |
| | rapid beeping for 2 minutes followed by a |
| | single beep every minute. |
| | 3. Press and release the pwr/prg button |
| | (720) to stop the beeping. |
| | 4. Remove the E-PAGE ™ 96 cassette (100) |
| | from the base (700, 710). |
| | 5. Open the gel cassette for staining or |
| | blotting applications. |
| Opening | Open the E-PAGE ™ 96 cassette Action (1310) with |
| the | the Butterfly Opener (included in the kit) |
| Cassette | (1320) to remove the gel. |
| (FIG. 13) | 1. Insert the wide side of the red |
| | Butterfly Opener (1320) between the tabs at |
| | the edge of the E-PAGE ™ 96 cassette (1310) |
| | and twist to separate the two halves of the |
| | cassette. |
| | 2. Pull apart the cassette halves (1330) |
| | with your hands until the cassette halves |
| | are separated. |
| | 3. Using a gel knife, trim the top and |
| | bottom electrode areas of the gel. |
| | 4. Proceed to staining or blotting. |
| Staining | Stain E-PAGE ™ 96 gels using any protein |
| and | staining method of choice. See below for |
| Blotting | more details. For blotting, see below. |
| Using | 1. Use an appropriate digital |
| E-Editor ™ | documentation system to capture a digital |
| 2.0 | image of the gel. |
| Software | 2. Download E-Editor ™ 2.0 software and |
| | the instruction manual for free on the |
| | internet at www.invitrogen.com/epage. |
| | 3. Use the E-Editor ™ 2.0 software to |
| | align and arrange the lanes in the image |
| | and save the reconfigured image for further |
| | analysis. |

### Exemplary Kit Contents

The kit contents for E-PAGE™ 96 Gels and E-PAGE™ Starter Kit are listed below:

TABLE 8

| | Quantity | |
|---|---|---|
| E-PAGE ™ 96 Gels | | |
| E-PAGE ™ 96 Gels | 8 | gels |
| E-PAGE ™ Loading Buffer 1 (4X) | 4.5 | ml |
| E-PAGE ™ Loading Buffer 2 (4X) | 4.5 | ml |
| Opener (1320) | 1 | |
| E-PAGE ™ Starter Kit | | |
| E-PAGE ™ 96 Gels (100) | 4 | gels |
| E-PAGE ™ Loading Buffer 1 (4X) | 4.5 | ml |
| E-PAGE ™ Loading Buffer 2 (4X) | 4.5 | ml |
| Opener (1320) | 1 | |
| Mother E-Base ™ (700) | 1 | |
| E-PAGE ™ SeeBlue ® Pre-stained Protein | 500 | μl |
| Standard | | |

The E-PAGE™ 96 Gels (100), loading buffers, and Mother E-Base™ (700) are shipped at room temperature. E-PAGE™ SeeBlue® Pre-stained Protein Standard is shipped on blue ice. Upon receipt, store E-PAGE™ 96 Gels and Mother E-Base™ (700) at room temperature. Do not allow the temperature to drop below 4° C. or rise above 40° C.

Store the E-PAGE™ Loading Buffers 1 and 2 at room temperature. After using the buffers, store at 4° C.

Store E-PAGE™ SeeBlue® Pre-stained Protein Standard at 4° C.

Each E-PAGE™ 96 Gel (100) contains 96 sample wells (130) and 8 marker wells (M) (145). Each cassette measures 13.5 cm (l)×10.8 cm (w)×0.67 cm (thick). The gel (110) comprises 6% polyacrylamide, at neutral pH, with a separation range of 10-300 kDa. The gel (110) is 3.7 mm thick, and the gel volume is 50 ml. The wells (130, 145) are 3 mm deep and measure 3.8 mm×1.8 mm at the well opening, and 3.3 mm×1.1 mm at the bottom of the well. The running distance (150) is 16 mm (one well to the next) and the spacing between wells is 9 mm.

The well openings of the E-PAGE™ 96 cassette are compatible with a multichannel pipettor or 8-, 12-, or 96-tip robotic loading devices.

### Product Qualification

E-PAGE™ 96 pre-cast Gels are qualified by running E-PAGE™ SeeBlue® Pre-Stained Protein Standards and BSA (bovine serum albumin) under standard running conditions as described in this manual. Gels are visualized for proper resolution, and migration of bands. Visual inspection is also performed to ensure that the gels are free from bubbles, spots, and any gel residues.

### E-Page™ Specifications and Procedure

E-PAGE™ High-Throughput (HTP) Protein Electrophoresis System is designed for fast, high-throughput protein electrophoresis in a horizontal format. The E-PAGE™ System consists of E-PAGE™ 96 Pre-cast Gels, E-Base™ Electrophoresis Device, E-PAGE™ Loading Buffers, and E-Editor 2.0 Software.

The E-PAGE™ HTP Protein Electrophoresis System is ideal for screening protein samples using these applications: staining (Coomassie®, silver, or fluorescent stains); Western blotting; in-gel staining using SYPRO® Orange; and functional assays.

### E-PAGE™ 96 Pre-Cast Gels

E-PAGE™ 96 Gels (100) are self-contained, pre-cast gels that include a buffered gel matrix and electrodes packaged inside a disposable, UV-transparent cassette.

Each E-PAGE™ 96 Gel contains 96 sample lanes and 8 marker lanes in a patented staggered well-format that is compatible with the standard 96-well plate format for automated robotic loading (see hereinabove for specifications).

After electrophoresis, the E-PAGE™ cassette (100) is easily opened with the opener (1320) included with the gel to remove the gel for staining or blotting applications.

In addition, each E-PAGE™ 96 cassette (100) is labeled with an individual barcode (180) to facilitate identification of the gel using commercial barcode readers.

### E-Base™

E-PAGE™ 96 Gels are used with a specially designed electrophoresis device of the present invention which is a base and a power supply all-in-one device. Two types of devices will be available from Invitrogen:

The Mother E-Base™ (700) (catalog no. EB-M03) has an electrical plug that can be connected directly to an electrical outlet and is used for electrophoresis of one E-PAGE™ 96 Gel.

The Daughter E-Base™ (710) (catalog no. EB-D03) connects to the Mother E-Base™ (700), and together they can be used for the electrophoresis of two or more E-PAGE™ 96 Gels. Note that the Daughter E-Base™ (710) does not have an electrical plug and cannot be used without a Mother E-Base™ (700), and that the E-PAGE™ 96 Gel is not compatible with the E-Gel® 96 mother base and daughter base available from Invitrogen Corp. (Carlsbad, Calif.) for use with agarose gels.

### Loading Buffers

The E-PAGE™ 96 Gels (100) are supplied with two loading buffers. The E-PAGE™ Loading Buffer 1 (4×) is optimized for E-PAGE™ 96 Gels, and is recommended for routine SDS-PAGE and staining or blotting applications.

The E-PAGE™ Loading Buffer 2 (4×) does not contain any SDS and is specifically designed for in-gel staining of proteins with SYPRO® Orange Protein Gel. Stain on E-PAGE™ 96 Gels.

### E-Editor™ 2.0 Software

The E-Editor™ 2.0 Software allows you to quickly reconfigure digital images of E-PAGE™ 96 results for analysis and documentation.

E-Editor™ 2.0 software will be downloadable for free from the Invitrogen Web site at www.invitrogen.com/epage, where a user can follow the instructions to download the software and user manual.

### Sample Preparation

Prepare your protein samples as described below for electrophoresis on E-PAGE™ 96 Gels. The E-PAGE™ 96 Gels contain SDS and are designed for performing electrophoresis under denaturing conditions. To obtain the best results, we recommend performing SDS-PAGE under reducing conditions. If you need to perform SDS-PAGE under non-reducing conditions, omit adding NuPAGE® Sample Reducing Agent (10×) during sample preparation.

### Materials Needed

Necessary materials include: protein sample; NuPAGE® Sample Reducing Agent (Invitrogen Corp., Carlsbad, Calif.); 4× E-PAGE™ Loading Buffer 1 (included in the kit); 4× E-PAGE™ Loading Buffer 2 for in-gel staining (included in the kit); SYPRO® Orange Protein Gel Stain (5000×) for in-gel staining (Molecular Probes, cat. no. S-6650); deionized water heating block set at 70° C.; and molecular weight markers.

Use up to 20 μg protein per well of the E-PAGE™ 96 Gel. The amount of protein will depend on the staining or western detection method used for visualizing proteins after electrophoresis. If you are unsure of how much to use, test a range of concentrations to determine the optimal concentration for your particular sample.

The maximum recommended protein load per well of the E-PAGE™ 96 Gel is 20 μg protein per well. Excess proteins will cause poor resolution.

To ensure a proper equilibrium of LDS (lithium dodecyl sulfate from Loading Buffer 1) to protein, limit sample protein or lipid (from the sample) amount to 20 μg per 10 μl of final sample volume.

The recommended total sample volume for E-PAGE™ 96 Gels is 10 μl. If desired, you may load between 5-20 μl of sample. Prior to sample loading, we recommend loading 10-20 μl deionized water first into all wells.

For best results, avoid loading less than 5 μl of sample and more than 20 μl of sample. See below for details on preparing samples.

### Loading Buffer

E-PAGE™ 96 Gels (100) are suitable for performing routine staining or blotting applications, and for in-gel staining using a fluorescent dye such as SYPRO® Orange (see below). Two types of loading buffers are supplied with E-PAGE™ 96 Gels. You need to use the appropriate loading buffer, based on the application as described below.

For SDS-PAGE and staining or blotting, we recommend using the 4× E-PAGE™ Loading Buffer 1 (included in your kit) for preparing samples. Preferably, do not use any other SDS-PAGE sample buffer. The E-PAGE™ Loading Buffer 1 is optimized for E-PAGE™ 96 Gels.

For in-gel staining with SYPRO® Orange on E-PAGE™ 96 Gels, use 4× E-PAGE™ Loading Buffer 2 (included in the kit). This loading buffer does not contain any SDS and is specifically designed for in-gel staining.

Proteins are pre-stained with the fluorescent dye, SYPRO® Orange, and separated on an E-PAGE™ 96 Gel. After electrophoresis, the gel cassette is placed on a standard UV transilluminator to view the fluorescent protein bands. There are no separate staining and destaining steps required.

Samples containing high salt or detergents will cause loss of resolution on E-PAGE™ 96 Gels. Dilute the samples such that the final concentration of the salt or detergent in the sample is <0.5% Triton® X-100; <0.5% Tween® 20; <4% SDS; <200 mM Tris; <250 mM NaCl.

To obtain the best results, we recommend using the protein molecular weight standards described herein (see Example 2, above). Use 10 μl of E-PAGE™ SeeBlue® Pre-stained Protein Standard or 5 μl of E-PAGE™ MagicMark™ Unstained Protein. Standard for western blotting.

### Preparing Samples for Routine Staining and Blotting

Use this protocol if you are performing SDS-PAGE followed by routine staining or blotting. Sample preparation for in-gel staining is on the next page.

If the E-PAGE™ Loading Buffer 1 (4×) is stored at 4° C., thaw the buffer to room temperature and mix briefly prior to use.

Prepare your samples in a total volume of 10 μl in the E-PAGE™ Loading Buffer 1 (4×) included in the kit as described below. If you need to prepare samples in a volume of 5-20 μl, adjust the volume accordingly.

TABLE 9

| Reagent | Reduced | | Non-reduced | |
|---|---|---|---|---|
| Protein Sample | x | μl | x | μl |
| E-PAGE ™ Loading Buffer 1 | 2.5 | μl | 2.5 | μl |
| (4X) | | | | |
| NuPAGE ® Sample Reducing | 1 | μl | — | |
| Agent (10X) | | | | |
| Deionized Water | to 10 | μl | to 10 | μl |

Heat the samples at 70° C. for 10 minutes. Store the E-PAGE™ Loading Buffer 1 (4×) at 4° C.

Proceed to Loading E-PAGE™ 96 Gels, below.

### Preparing Samples for In-Gel Staining

In-gel staining is a method of staining proteins with fluorescent dyes prior to electrophoresis. After electrophoresis, the proteins are easily visualized using a standard UV transilluminator or an imaging system without the need for any staining or destaining steps.

Use this protocol for in-gel staining with SYPRO® Orange Protein Gel Stain.

For in-gel staining, the final protein concentration after dilution (step 5, below) must be at least 200 ng/protein band to obtain good detection. Be sure to follow the protocol exactly as described below. In-gel staining is recommended with partially purified protein samples, as lipids and other components from a cell lysate may interfere with the stain or detection. Thaw the E-PAGE™ Loading Buffer 2 (4×) to room temperature, if stored at 4° C., and mix briefly prior to use

Prepare fresh SYPRO® Orange Stain Concentrate by mixing 10 ml SYPRO® Orange Protein Gel Stain, 5000× (Molecular Probes, cat. no. S-6650) with 50 ml 4× E-PAGE™ Loading Buffer 2 (included in the kit) and 140 ml deionized water.

Prepare your samples in the E-PAGE™ Loading Buffer 1 (included in the kit) as described in Table 10.

TABLE 10

| Reagent | Reduced | Non-reduced |
|---|---|---|
| Protein Sample: | x; l | x; l |
| E-PAGE ™ Loading | 2.5; l | 2.5; l |
| Buffer 1 (4X): | | |
| NuPAGE ® Sample | 1.0; l | — |
| Reducing Agent | | |
| (10X): | | |
| Deionized Water: | to 10; l | To 10; l |

Heat the samples at 70° C. for 10 minutes, and then cool the samples to room temperature.

Add 5 ;l of Stain Concentrate from Step 1 to 10 ;l of sample from Step 2. Mix and incubate the tube for 10 minutes at room temperature.

To 15 ml of sample from Step 4, add 25 ;l 4× E-PAGE™ Loading Buffer 2 and 60 ;l deionized water. Mix well. You will load 5-20 ;l per well of this final sample.

Load the E-PAGE™ 96 Gel as follows. The Mother E-Base™ (700) and Daughter E-Base™ (710) are designed to fit most robotic platforms allowing you to load and run E-PAGE™ 96 Gels directly on the robot.

If you need to load multiple gels on a robotic platform while other gels are running on the E-Base™, use an E-Holder™ Platform.

If you are using an automated liquid handling device, it is important to align the robotic tip loading assembly to the proper setting prior to loading samples on the E-PAGE™ 96 Gel. This ensures proper loading of samples into the wells.

Each E-PAGE™ 96 Gel (100) is labeled with an individual barcode (with a number) (180). The barcode facilitates identification of each gel cassette during electrophoresis of multiple gels. Each E-PAGE™ 96 Gel contains an EAN 39 type of barcode, which is recognized by the majority of commercially available barcode readers. Refer to the manufacturer's instructions to set up the barcode reader.

When capturing an image of the E-PAGE™ 96 Gel, note that the barcode label (180) is easily overexposed. To ensure that the barcode label is distinct and readable in the image, experiment with different shutter settings for your particular camera.

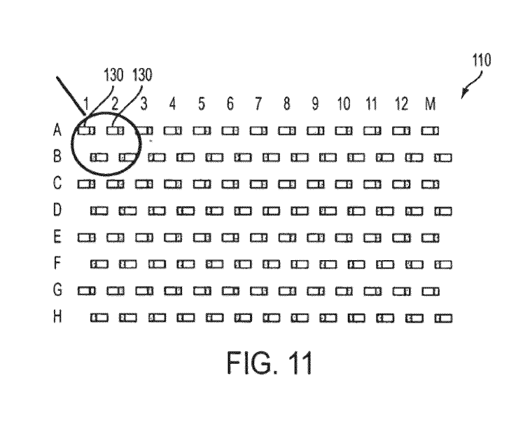

The wells (130) of the E-PAGE™ 96 Gel (100) are staggered to provide maximum run length (FIG. 11). For proper sample loading, it is important to program your robotic loading system to set the A1 tip of the 8-, 12-, or 96-tip robotic head over the E-PAGE™ 96 Gel as described below.

Set the position of the first tip (1210), approximately 1 mm above the slope (1225) of the A1 well (1220) (FIG. 12). This will ensure that the remaining tips (1260) are aligned above the slopes (1250) of the remaining wells (1230). Refer to the manufacturer's Manual of your robot to program this setting. After programming the setting, load your samples (1240). During loading, the samples will fall onto the slopes of the wells and be drawn into the wells by capillary force.

The recommended run time for E-PAGE™ 96 Gels is 14 minutes.

You will need to select an appropriate program on the base prior to inserting a gel into the base, as follows. Plug the Mother E-Base™ (700) into an electrical outlet using the electrical plug (750) on the base. If using Daughter E-Base™ (710), connect the Daughter E-Base™ (710) to a Mother E-Base™ (700) or another Daughter E-Base™ (710) connected to a Mother E-Base™ (700). The display (735) will show EP or last program used (EG or EP).

Select the program EP for E-PAGE™ 96 Gels by pressing and releasing the pwr/prg (power/program) (720) button. The digital display (735) will show EP. To obtain the best results, run the E-PAGE™ 96 Gel (100) immediately after removal from the pouch and loading. Store and run E-PAGE™ 96 Gels at room temperature. Always load 10-20 μl deionized water first into all wells (130, 145) prior to sample loading.

For optimal results, we do not recommend running reduced and non-reduced samples on the same gel. If you do choose to run these samples on the same gel, avoid running reduced and non-reduced samples in adjacent lanes as the reducing agent may have a carry-over effect on the non-reduced samples if they are in close proximity.

Avoid running samples containing different salt or protein concentrations in adjacent lanes.

### Loading E-PAGE™ Gels

Each E-PAGE™ 96 Gel (100) is supplied individually wrapped and ready for use. Use short, rigid tips for loading. Open the package and remove the E-PAGE™ 96 Gel. Remove the plastic comb from the gel. Slide the gel into the two electrode connections (160, 170) on the Mother (700) or Daughter E-Base™ (710). The two copper electrodes (160, 170) on the right side of the cassette (100) must be in contact with the two electrode connections (740) on the base (700), as shown in FIG. 9.

Load samples into the gels using a multichannel pipettor or a liquid handling system. Load deionized water to each well (130, 145) of the E-PAGE™ 96 Gel prior to loading your samples or protein molecular weight standard as described below. If loading 5-10 μl of sample in loading buffer, first load 20 μl of deionized water. If loading 11-20 μl of sample in loading buffer, first load 19-10 μl deionized water.

Load the appropriate protein molecular weight marker in the marker wells of the gel.

Proceed immediately to electrophoresis.

### Electrophoresis of E-PAGE™ 96 Gels

After loading your protein samples on the E-PAGE™ 96 Gels (100), proceed immediately to electrophoresis using the E-Base™. The default run time for the E-PAGE™ 96 Gel is 14 minutes.

Instructions for running an E-PAGE™ 96 Gel in a Mother E-Base™ (700) or Daughter E-Base™ (710) are provided below. For more details on setting the timer or interrupting a run, refer to Example 7, above.

It is not necessary to have a gel in the Mother E-Base™ (700) if you are using a Daughter E-Base™ (710). However, the Mother E-Base™ (700) must be plugged into an electrical outlet.

To begin electrophoresis, press and release the pwr/prg (power/program) button (720) located on the lower right corner of the Mother E-Base™ (700) or Daughter E-Base™ (710). If you are using a Daughter E-Base™ (710), press and release the pwr/prg button (720) located on the lower right corner of the Daughter E-Base™ (710). The red light (730) will change to a green light and the digital display (735) will show the count down time during the run.

While the run is in progress, you can add to the run time by pressing the timer button to select the desired time and then release the timer button. Avoid running an E-PAGE™ 96 Gel for more than 30 minutes.

If your sample contained high salt or detergent concentrations, you may need to manually increase the run time.

The Mother E-Baser (700) and Daughter E-Base™ (710) will signal the end of the run with a flashing red light (730) and rapid beeping for 2 minutes followed by a single beep every minute.

At the end of the run, the digital display (735) will show the original time setting (not any time change that was made during the electrophoresis). The digital display (735) will also show the elapsed time (up to 19 minutes with a negative sign) since the end of the run.

Press and release the pwr/prg button (720) to stop the beeping. The light will turn to a steady red and the digital display will show the last time setting.

Remove the gel cassette from the Mother E-Base™ (700) and Daughter E-Base™ (710). Note that the bands in the gel will diffuse within 40 minutes.

For in-gel staining, place the cassette on a standard UV transilluminator or an imaging system to capture an image of the gel. The maximum excitation wavelengths for SYPRO® Orange Protein Gel. Stain are at 300 nm and 470 nm, and maximum emission wavelength is at 570 nm. In-gel staining with SYPRO® Orange Protein Gel Stain will result in fluorescent protein bands.

### Interrupting an Electrophoresis Run

You can interrupt an electrophoresis run at any time by pressing and releasing the pwr/prg button (720) to stop the current. The stopped current is indicated by a steady red light (730), and the digital display (735) will flash to indicate that the run has been interrupted.

You can remove the gel from the base to check the progress of the run. Then, to continue the run from the point at which it was stopped, reinsert the gel and press and release the pwr/prg button (720). The light (730) changes to steady green and the digital display shows the count down time. Alternatively, to cancel the rest of the interrupted run, press and hold the pwr/prg button (720) for a few seconds. The digital display will reset and the base will return to Ready Mode. If desired, you can then program a new run time and rerun the gel.

In case of an external power failure, the run will continue when the power resumes. The Mother E-Base™ (700) and Daughter E-Base™ (710) will signal the end of the run as described on the previous page, except the light (720) will be an alternating red/green to indicate that an external power failure has occurred during the run.

The surfaces of the Mother E-Base™ (700) and Daughter E-Base™ (710) should be kept free of contaminants. To clean, disconnect bases from power source and wipe clean with a dry cloth. Do not attempt to open or service the bases.

### Opening the E-PAGE™ 96 Cassette

Prior to staining or blotting the E-PAGE™ 96 Gel, you need to open the cassette using the red, plastic Butterfly Opener to remove the gel, as shown in FIG. 13.

Insert the wide side of the red Butterfly Opener (included in the kit) (1320) between the tabs at the edge of the E-PAGE™ 96 cassette (1310) and twist to separate the two halves of the cassette (FIG. 13A) (1330). Gently pull apart the cassette halves (1330) with your hands until the cassette halves are completely separated and the gel is exposed (FIG. 13B). Carefully remove the gel from the cassette. Using a gel knife, trim the top and bottom electrode areas of the gel.

If you are using a mini-cell blot module (XCell II™ Blot Module), you may need to cut the gel into 2 halves so the gel can fit into the blot module. You will need 2 units of the XCell II™ Blot Module on hand before proceeding for transfer. For a standard blot module that can fit a large format gel, there is no need to cut the gel into 2 halves.

Proceed to staining or blotting.

### Staining E-PAGE™ 96 Gels

Stain E-PAGE™ 96 Gels using any method of choice. Since E-PAGE™ 96 Gels are thicker than most SDS-PAGE mini-gels, you may need to optimize the staining and destaining steps. Instructions for staining E-PAGE™ 96 Gels using Coomassie® stain, fluorescent stain, SilverXpress® Silver Stain, and SimplyBlue™ SafeStain are described in this section.

Note that small pieces of gel material may remain in the wells of an E-PAGE™ Gel after removal of the gel from the cassette. To obtain the best staining results, remove any small pieces of gel material in the wells of the gel by gently rubbing a gloved hand over the well side of the gel.

### Materials Needed

Necessary materials include SimplyBlue™ SafeStain or Coomassie® R-250 Stain and staining trays. Silver staining of gels also requires reagent grade methanol and acetic acid, SilverXpress® Silver Staining Kit and ultra pure water (18 mega-Ohm/cm recommended). Fluorescent gel staining requires either SYPRO® Orange (cat. no. S-6650) or SYPRO® Ruby (cat. no. S-12001) Protein Gel Stains, available from Molecular Probes (Eugene Oreg., USA).

You may use any Coomassie® R-250 staining protocol of choice. To obtain the best results with E-PAGE™ 96 Gels, we recommend a longer time for destaining step as E-PAGE™ gels are thicker than standard protein mini-gels.

The E-PAGE™ 96 Gels can be stained with fluorescent protein stains such as SYPRO® Orange or SYPRO® Ruby Protein Gel Stains available from Molecular Probes.

After electrophoresis, remove the gel from the cassette (as per above) and follow manufacturer's recommendations for staining and visualizing the gel.

### Silver Staining

A brief protocol for staining E-PAGE™ 96 Gel with SilverXpress® Silver Staining Kit is provided at Table 11. For details, refer to the manual available on the internee at www.invitrogen.com. Note that the SilverQuest™ Silver Staining protocol has not been optimized for use with E-PAGE™ 96 Gels.

For all staining and washing steps described below, be sure to use sufficient volume of reagent to completely cover the gel using a suitable container such that the gel moves freely during the staining and washing steps. Perform all steps on a rotary shaker set at 1 revolution per second.

TABLE 11

| Step | Reagent | | Procedure |
|---|---|---|---|
| Fix | Ultrapure water | 90 ml | Fix the gel in fixing |
| | Methanol | 100 ml | solution for 40 minutes. |
| | Acetic Acid | 20 ml | Decant the solution. |
| | Final Volume | 200 ml | |
| Sensitize | Ultrapure water | 105 ml | Incubate the gel in 2 |
| | Methanol | 100 ml | changes of Sensitizing |
| | Sensitizer | 5 ml | Solution for 60 minutes, |
| | Final Volume | 200 ml | each. Decant the |
| | | | solution |
| Wash | Ultrapure water | | Wash the gel twice with |
| | | | ultrapure water for 30 |
| | | | minutes each. |
| Stain | Stainer A | 5 ml | Incubate the gel in |
| | Stainer B | 5 ml | Staining Solution for 60 |
| | Ultrapure water | 90 ml | minutes. Decant the |
| | Final Volume | 100 ml | solution. |
| Wash | Ultrapure water | | Wash the gel twice with |
| | | | ultrapure water for 30 |
| | | | minutes each. |
| Develop | Developer | 5 ml | Incubate the gel in |
| | Ultrapure water | 95 ml | Developing Solution for |
| | Final Volume | 100 ml | 3-30 minutes. |
| Stop | Stopping Solution | | Add the Stopping |
| | | | Solution directly to the |
| | | | Developing Solution when |
| | | | the desired staining |
| | | | intensity is reached. |
| | | | Incubate the gel in |
| | | | Stopping solution for 10 |
| | | | minutes. Decant the |
| | | | solution. |
| Wash | Ultrapure water | | Wash the gel three times |
| | | | with ultrapure water for |
| | | | 15 minutes each. |

### SimplyBlue™ SafeStain

Brief protocols for staining E-PAGE™ 96 Gel with SimplyBlue™ SafeStain are described below. For details on SimplyBlue SafeStain™, refer to the manual available on the internet at www.invitrogen.com.

For all staining and washing steps described below, be sure to use sufficient water or stain to completely cover the gel using a suitable container such that the gel moves freely during the staining and washing steps.

For routine staining, use Protocol A. To obtain the clearest background for photography, use Protocol B, which includes a 12-24 h washing step to improve the background.

Protocol A. Place the gel in an appropriate container. Rinse the gel 3 times for 5 minutes each with deionized water to remove SDS and buffer salts. Stain the gel with sufficient SimplyBlue™ SafeStain to cover the gel. Incubate at room temperature for 1.5 h with gentle shaking. Decant the stain. Wash the gel with deionized water for 3 h with intermittent water changes.

To obtain the clearest background for photography, use Protocol B. Fix the gel in 20% acetic acid for 30 minutes at room temperature. Decant acetic acid. Stain the gel with sufficient SimplyBlue™ SafeStain for 30 minutes at room temperature with gentle shaking. Decant the stain and rinse the gel briefly with deionized water. Wash the gel in deionized water for 12-24 h at room temperature with one water change

You may dry the stained E-PAGE™ Gel by vacuum drying or air-drying. We recommend using the Large Gel Drying Kit available from Invitrogen to air-dry the gel. Refer to the Large Drying Kit manual for details. Note that the E-PAGE™ 96 Gel will need at least 4 days for complete drying. If you are using vacuum drying, follow the manufacturer's instructions.

### Blotting E-PAGE™ 96 Gels

In one embodiment of the present invention, E-PAGE™ 96 Gels are blotted as described in Table 12.

TABLE 12

| | |
|---|---|
| | Instructions for blotting E-PAGE ™ 96 Gels |
| | using the XCell II ™ Blot Module are |
| | described in this section. For detailed |
| | instructions on the XCell II ™ Blot Module, |
| | refer to the manual (IM-9051). This manual |
| | is available for downloading from |
| | www.invitrogen.com. |
| | If you are using other blotting apparatus, |
| | refer to the manufacturer's |
| | recommendations. |
| Materials | You will need the following items. |
| Needed | NuPAGE ® Transfer Buffer |
| | NuPAGE ® Antioxidant |
| | XCell II ™ Blot Module (2 units required for |
| | transfer of 1 E-PAGE ™ 96 Gel, see Important |
| | on next page) |
| | XCell SureLock ™ Mini-Cell |
| | Novex ® pre-cut membrane/filter paper |
| | sandwiches |
| Preparing | We recommend using the NuPAGE ® Transfer |
| Transfer | Buffer for optimal transfer of proteins |
| Buffer | from the E-PAGE ™ 96 Gel. The NuPAGE ® |
| | Antioxidant is used in the transfer buffer |
| | for blotting reduced proteins and prevents |
| | the proteins from reoxidizing. |
| | Prepare 1000 ml of 1X NuPAGE ® Transfer |
| | Buffer using the NuPAGE ® Transfer Buffer |
| | (20X) as follows: |

| | | |
|---|---|---|
| | Reduced | Non-Reduced |
| Component | Sample | Sample |
| NuPAGE ® | 50 ml | 50 ml |
| Transfer | | |
| Buffer (20X) | | |
| NuPAGE ® | 1 ml | — |
| Antioxidant | | |
| Methanol | 100 ml | 100 ml |
| Deionized | 849 ml | 850 ml |
| Water | | |
| Total Volume | 1000 ml | 1000 ml |

| | |
|---|---|
| | NuPAGE ® Transfer Buffer with 10% methanol |
| | provides optimal transfer of an E-PAGE ™ 96 |
| | Gel in the blot module. |
| Preparing | 1. Use 700 ml 1X NuPAGE ® Transfer Buffer |
| Blotting | to soak the blotting pads until |
| Pads and | saturated. Remove air bubbles by |
| Membrane | squeezing the blotting pads while they |
| | are submerged in buffer. |
| | 2. Prepare Novex ® pre-cut membrane/filter |
| | paper sandwiches as described below. |
| | PVDF membrane: Pre-wet the PVDF |
| | membrane for 30 seconds in methanol, |
| | ethanol, or isopropanol. Briefly rinse |
| | in deionized water and place the |
| | membrane in a shallow dish containing |
| | 50-100 ml 1X NuPAGE ® Transfer Buffer |
| | for several minutes. |
| | Nitrocellulose membrane: Place the |
| | membrane directly into a tray |
| | containing 1X NuPAGE ® Transfer Buffer |
| | for several minutes. |
| | Filter paper: Soak briefly in 1X |
| | NuPAGE ® Transfer Buffer immediately |
| | before using. |
| | 3. Use the gel immediately following the |
| | run. Do not soak the gel in transfer |
| | buffer. You will need to cut the gel |
| | into 2 halves so the gel can fit into the |
| | XCell II ™ Blot Module. |
| | To obtain the best transfer, we recommend |
| | transferring only one half of E-PAGE ™ 96 |
| | Gel in one XCell II ™ Blot Module. |
| | Inefficient transfer may occur if you |
| | transfer 2 halves of the E-PAGE ™ 96 Gel in |
| | one blot module at the same time as the E- |
| | PAGE ™ 96 Gel is thicker than regular mini- |
| | gels. |
| | If you are using the XCell II ™ Blot Module |
| | for transferring 1 E-PAGE ™ 96 Gel, we |
| | recommend that you have two XCell II ™ Blot |
| | Modules on hand before proceeding for |
| | transfer. |
| | If you are using a standard blot module |
| | that can fit a large format gel, you will |
| | need only 1 blot module. |
| Blotting | 1. Remove any small pieces of gel from the |
| Procedure | wells by gently rubbing a gloved finger |
| | over the well side of the gel. Presence of |
| | gel pieces may cause bubbles during the |
| | gel/membrane assembly. |
| | 2. Lay the gel on a flat surface, well |
| | side up, in a suitable container and pour |
| | sufficient 1X NuPAGE ® Transfer buffer over |
| | the gel to fill all wells of the gel. |
| | 3. Place a piece of pre-soaked filter |
| | paper on top of the gel and remove any |
| | trapped air bubbles by gently rolling a |
| | glass pipette over the filter paper. |
| | 4. Turn the gel over so the filter paper |
| | and gel and are facing downwards over a |
| | gloved hand or clean flat surface |
| | 5. Place a pre-soaked transfer membrane on |
| | the gel and remove any trapped air bubbles. |
| | 6. Place another pre-soaked filter paper |
| | on top of the membrane and remove any |
| | trapped air bubbles. |
| | 7. Place two pre-soaked blotting pads into |
| | the cathode core of the blot module. |
| | 8. Place the gel/membrane assembly on the |
| | blotting pads, such that the gel is closest |
| | to the cathode core. |
| | 9. Add another pre-soaked blotting pad on |
| | top of the membrane assembly. |
| | 10. Add enough pre-soaked blotting pads to |
| | rise to 0.5 cm over rim of cathode core. |
| | Place the anode (+) core on top of the |
| | pads. |
| | 11. Hold the blot module together firmly |
| | and slide it into the guide rails on the |
| | Lower Buffer Chamber. |
| | 12. Insert the Gel Tension Wedge into the |
| | Lower Buffer Chamber and lock the wedge |
| | into position. |
| | 13. Fill the blot module with 1X NuPAGE ® |
| | Transfer Buffer until the gel/membrane |
| | assembly is just covered. |
| | 14. Fill the Outer Buffer Chamber with 650 ml |
| | deionized water. |
| | 15. Place the lid on the unit and connect |
| | electrical leads to the power supply. |
| | 16. Perform transfer for nitrocellulose or |
| | PVDF membranes using 35 V constant for 1 |
| | hour. The expected start current is 170 mA. |

### Using E-Holder™ Platform

In one embodiment of the present invention, the E-Holder™ Platform is used as described in Table 13.

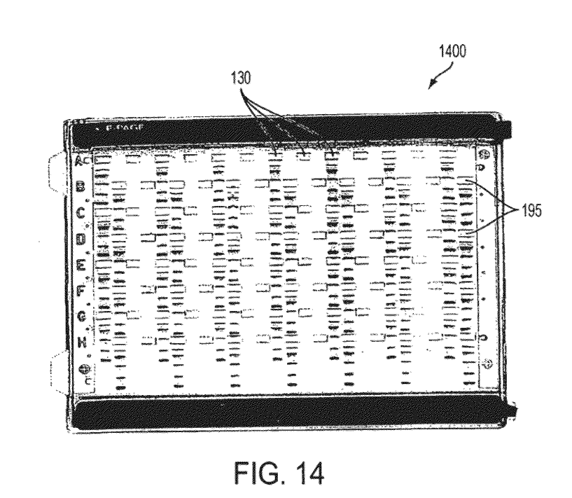

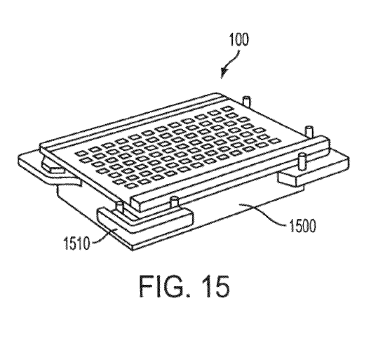

TABLE 13

| | |
|---|---|
| Introduction | The E-Holder ™ Platform is designed to hold |
| | E-PAGE ™ 96 Gels during robotic loading. Use |
| | the E-Holder ™ (1500) when you need to load |
| | multiple gels on a robotic platform while |
| | the other gels are running on the E-Base ™ |
| | Note: The E-Holder ® (1500) is not a power |
| | supply unit, cannot be connected to an |
| | electrical outlet, and cannot be used to |
| | run E-PAGE ™ 96 Gels. |
| | To obtain the best results, run E-PAGE ™ 96 |
| | Gels on the Mother E-Base ™ (700) or |
| | Daughter E-Base ™ (710) within 15 minutes |
| | after loading on E-Holder ™ (1500). |
| Procedure | 1. Place the E-Holder ™ (1500) on the robotic |
| | platform. |
| | 2. Open the package and remove the gel. |
| | 3. Remove the comb from the E-PAGE ™ 96 |
| | cassette (100). |
| | 4. Place the E-PAGE ™ 96 cassette in the E- |
| | Holder ™ (1500). Align the bottom left end |
| | of the cassette in the lower left |
| | alignment corner (1510) of the E-Holder ™ |
| | (1500) as shown in FIG. 15. |
| | 5. Note: The E-PAGE ™ 96 Gel will not fit |
| | into the E-Gel ® 96 holder previously |
| | available from Invitrogen due to the tabs |
| | on the E-PAGE ™ 96 cassette. |
| | 6. Set up your robotic system to load |
| | samples into the gel placed on an E- |
| | Holder ™ (1500) as described on page 10. |
| | Program your robotic system to load the |
| | samples approximately 5 minutes before |
| | the previous electrophoresis run is |
| | complete. This will ensure that the |
| | loaded gel from the E-Holder ™ (1500) will |
| | be placed onto a Mother E-Base ™ (700) or |
| | Daughter E-Base ™ (710) within the |
| | recommended time of 15 minutes. |
| Expected | 7. Results obtained using a 6% E-PAGE ™ 96 |
| Results | Gel (1400) are shown in FIG. 14. |
| | 8. E-PAGE ™ SeeBlue ® Pre-stained Protein |
| | Standard (10 μl) was loaded into |
| | alternate sample wells and 8 marker |
| | wells. The gel was electrophoresed for |
| | 14 minutes using standard conditions |
| | described in this manual and imaged using |
| | flat bed scanner from Hewlett Packard. |
| | 9. Note: The wells (130, 145) of an E-PAGE ™ |
| | 96 Gel (100) are staggered. Protein bands |
| | migrate between adjacent wells in the row |
| | below. For example, the bands of lane A3 |
| | will migrate between wells B2 and B3. |
| | 10. Western blotting results obtained |
| | using a 6% E-PAGE ™ 96 Gel are shown |
| | below. |
| | 11. E-PAGE ™ MagicMark ™ Unstained Protein |
| | Standard (5 μl) was loaded onto some |
| | sample wells (see image below). The gel |
| | was electrophoresed for 14 minutes using |
| | standard conditions described in this |
| | manual. Proteins were transferred to a |
| | 0.45 μm Nitrocellulose membrane at 35 V |
| | for 60 minutes with 1X NuPAGE ® Transfer |
| | buffer with 10% methanol using the XCell |
| | II ™ Blot Module. Detection was performed |
| | with WesternBreeze ® Anti-Mouse |
| | Immunodetection Kit using 1:5000 dilution |
| | of Anti-V5 primary antibody from |
| | Invitrogen. The image was captured with a |
| | Fuji LAS-1000 Luminometer using a 20 |
| | second exposure. See FIG. 6 |

### Using E-Editor™ 2.0 Software

In one embodiment of the present invention, E-Editor 2.0 Software is used as described in Table 14.

TABLE 14

| | |
|---|---|
| | E-Editor 2.0 software for Windows ® allows |
| | you to reconfigure digital images of E- |
| | PAGE ™ 96 Gels for analysis and |
| | documentation. The staggered lanes in an E- |
| | PAGE ™ 96 Gels are difficult to compare and |
| | analyze by standard 1-D gel analysis |
| | programs such as Bio-Rad's Quantity One, |
| | Phoretix 1D, or Kodak 1D software. E- |
| | Editor ™ 2.0 software reconfigures the wells |
| | of an E-PAGE ™ 96 Gel into a side-by-side |
| | format for easy comparison and analysis. |
| | You can reconfigure gels that were scanned |
| | in the original gel cassette, or gels that |
| | were removed from the cassette and stained |
| | or blotted. You can also group the images |
| | of multiple gels loaded from a 384-well |
| | microtiter plate into a single image with a |
| | layout corresponding to that of the |
| | original plate. |
| | Capture an image of the gel as described |
| | below and then, use the E-Editor 2.0 |
| | software to: |
| | Align and arrange the lanes in the image |
| | Save the reconfigured image for further |
| | analysis |
| | Copy and paste selected lanes or the |
| | entire reconfigured image into other |
| | applications for printing, saving, e- |
| | mailing, and/or publishing on the Web |
| Imaging | Use an appropriate gel documentation system |
| the Gel | to capture a digital image of the gel. |
| | When imaging, the gel should be properly |
| | aligned (i.e., not at an angle) and gel |
| | features should be clear and distinct. |
| | Proceed to Downloading Software. |
| Downloading | E-Editor 2.0 software can be downloaded for |
| Software | free from the Invitrogen Web site. Go to |
| | www.invitrogen.com/epage and follow the |
| | instructions to download the software and |
| | user manual. |
| | The E-Editor ™ 2.0 software is available as |
| | Windows ® compatible software. The |
| | Macintosh ® compatible version of the |
| | software will be available soon. However, |
| | if the E-PAGE ™ 96 Gel is not removed from |
| | the cassette, you can use the Macintosh ® |
| | version of the software for reconfiguring |
| | the image. |

### Troubleshooting

In one embodiment of the present invention, troubleshooting of problems is performed as described in Table 15.

### Troubleshooting

The table below provides some solutions to possible problems you might encounter during the electrophoresis of E-PAGE™ 96 Gels.

For troubleshooting problems with E-Base™, refer to the manual supplied with the E-Base™.

TABLE 15

| Problem | Cause | Solution |
|---|---|---|
| No | Daughter E- | Do not use the Daughter E- |
| current | Base ™ (710) | Base ™ (710) without a Mother |
| | used without | E-Base ™ (700). The Daughter |
| | a Mother E- | E-Base ™ (710) does not have |
| | Base ™ (700) | an electrical plug to connect |
| | | to an electrical outlet. |
| | Copper | Make sure that the copper |
| | contacts in | contacts in the base are |
| | the base | intact. |
| | (740) are | |
| | damaged due | |
| | to improper | |
| | use | |
| | Expired or | Use properly stored gels |
| | defective gel | before the specified |
| | cassette used | expiration date. |
| | E-PAGE ™ 96 | Remove cassette and reinsert. |
| | cassette | When the cassette is |
| | (100) is not | correctly inserted and power |
| | correctly | is on, a fan in the base will |
| | inserted into | begin to run and a steady red |
| | base (700, | light (730) will be |
| | 710) | illuminated on the base. |
| Poor | Sample is | Do not load more than 20 μg |
| resolution | overloaded | of protein sample per well. |
| or | | For in-gel staining, load at |
| smearing | | least 200 ng protein per |
| of bands | | band. |
| | Very low | Load the recommended sample |
| | volumes of | volume of 5-20 μl and always |
| | sample were | load 10-20 μl deionized water |
| | loaded | in all wells prior to sample |
| | | loading. |
| | | Avoid introducing bubbles |
| | | while loading the samples. |
| | | Bubbles will cause band |
| | | distortion. |
| | | For proper band separation, |
| | | we recommend keeping sample |
| | | volumes uniform and loading |
| | | deionized water into empty |
| | | wells. |
| Poor | Incorrect | Use only E-PAGE ™ Loading |
| resolution | loading | Buffer 1 or 2 with E-PAGE ™ 96 |
| or | buffer used | gels. Do not use any other |
| smearing | | SDS-PAGE sample buffers. |
| of bands | High salt or | Be sure the final |
| | detergent | concentration of salt or |
| | concentration | detergent in the sample is as |
| | in samples | described on page 7. You may |
| | | need to manually increase the |
| | | run time for high salt or |
| | | detergent samples to obtain |
| | | optimal results. |
| | Gel was not | For best results, the gel |
| | electrophoresed | should be run within 15 |
| | immediately | minutes of sample loading. |
| | after sample | |
| | loading | |
| | A1 tip not | Be sure to align the A1 tip |
| | aligned | properly prior to loading |
| | | your samples using automated |
| | | robotic loading (page 10). |
| | Expired gel | Use fresh gels. Use properly |
| | used | stored gels before the |
| | | specified expiration date. |
| Sample | Sample is | Be sure to load the |
| leaking | overloaded | recommended volume of sample |
| from the | | per well (page 6). |
| wells | | Remove the comb carefully |
| | | without damaging the wells. |
| Over-run | Accidentally | Select program EP for E-PAGE ™ |
| the gel | selected an | 96 Gels. |
| or need | incorrect | If you accidentally selected |
| more time | program | an incorrect program and are |
| to run | | at the beginning of the run, |
| gel | | stop the run and select the |
| | | desired program. |
| | | If you are well into the run, |
| | | check the gel to see where |
| | | the loading dye is running. |
| | | Estimate the amount of time |
| | | remaining and then manually |
| | | stop the run. |

### Accessory Products

In one embodiment of the present invention, one or more of the accessory products listed in Table 16 is used in conjunction with a method or apparatus of the present invention.

TABLE 16

| | |
|---|---|
| Additional | Additional products that may be used with |
| Products | E-PAGE ™ 96 HTP Protein Electrophoresis |
| | System are available separately from |
| | Invitrogen. Ordering information is |
| | provided below. |

| Product | Quantity | | Catalog no. |
|---|---|---|---|
| Mother E-Base ™ (700) | 1 | | EB-M03 |
| Daughter E-Base ™ (710) | 1 | | EB-D03 |
| E-Holder ™ Platform (1500) | 2 | | G7300-01 |
| E-PAGE ™ Loading Buffer 1 | 4.5 | ml | EPBUF-01 |
| (4X) | | | |
| E-PAGE ™ Loading Buffer 2 | 4.5 | ml | EPBUF-02 |
| (4X) | | | |
| NuPAGE ® Sample Reducing | 10 | ml | NP0009 |
| Agent (10X) | | | |
| E-PAGE ™ SeeBlue ® Pre-stained | 500 | μl | LC5700 |
| Protein Standard | | | |
| E-PAGE ™ MagicMark ™ Unstained | 250 | μl | LC5701 |
| Protein Standard | | | |
| NuPAGE ® Transfer Buffer | 125 | ml | NP0006 |
| (20X) | | | |
| NuPAGE ® Antioxidant | 15 | ml | NP0005 |
| XCell SureLock ™ Mini-Cell & | 1 | unit | EI0002 |
| XCell II ™ Blot Module | | | |
| Nitrocellulose Membrane, | 20 | blots | LC2001 |
| 0.45 μm | | | |
| Invitrolon ™ PVDF membranes, | 20 | blots | LC2005 |
| 0.45 μm | | | |
| SimplyBlue ™ SafeStain | 1 | L | LC6060 |
| SilverXpress ® Silver | 1 | kit | LC6100 |
| Staining Kit | | | |
| Large Gel Drying Kit | 1 | kit | NI2207 |
| Gel-Dry ™ Drying Solution | 500 | ml | LC4025 |
| (1X) | | | |
| WesternBreeze ® Chromogenic | | | |
| Kit | | | |
| Anti-Mouse | 1 | kit | WB7103 |
| Anti-Rabbit | 1 | kit | WB7105 |
| Anti-Goat | 1 | kit | WB7107 |
| WesternBreeze ® | | | |
| Chemiluminescent Kit | | | |
| Anti-Mouse | 1 | kit | WB7104 |
| Anti-Rabbit | 1 | kit | WB7106 |
| Anti-Goat | 1 | kit | WB7108 |

The invention illustratively described herein suitably may be practiced in the absence of any element or elements, limitation or limitations, which is not specifically disclosed herein. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed herein, optional features, modification and variation of the concepts herein disclosed may be resorted to by those skilled in the art, and that such modifications and variations are considered to be within the scope of this invention as defined by the appended claims. In addition, where features or aspects of the invention are described in terms of Markush groups, those skilled in the art will recognize that the invention is also thereby described in terms of any individual member or subgroup of members of the Markush group.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other aspects of the invention are within the following claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1-34. (canceled)

35. A composite electrophoretic separation medium comprising:
a first gel portion and a second gel portion, said first and second gel portions comprising agarose, linear polyacrylamide, and a buffering system; and
a resolving portion positioned between the first and second gel portions, said resolving portion comprising agarose, polyacrylamide, a buffering system and a photoinitiator, said resolving portion lacking linear polyacrylamide.

36. The composite electrophoretic separation medium according to claim 35, wherein the concentration of agarose in the first gel portion, the second gel portion or the resolving portion is less than about 5%.

37. The composite electrophoretic separation medium according to claim 35, wherein the concentration of agarose in the first gel portion, the second gel portion or the resolving portion is between 1% to 2%.

38. The composite electrophoretic separation medium according to claim 35, wherein the concentration of polyacrylamide in the resolving portion is between about 1% to about 8.5%.

39. The composite electrophoretic separation medium according to claim 35, wherein the buffer system comprises at least one of bistris, tricine, BES (2-[bis(2-hydroxyethyl)am-ino]ethanesulfonic acid), MOPS (3-[N-Morpholino]propanesulfonic acid), or MES (2-(N-Morpholino)ethanesulfonic acid).

40. The composite electrophoretic separation medium according to claim 35, wherein the photoinitiator is selected from the list 1-hydroxy-cyclohexyl-phenyl-ketone (1-HCPK), 2,2-dimethoxy-2-phenylacetophenone, 2-methyl-1-[4-(methylthio)phenyl]-2-(4-morpholinyl)-1-propanone, 2-hydroxy-2-methyl-1-phenyl-1-propanone, 4-(2-hydroxyethoxy)phenyl]-2-(hydroxy-2-propyl)ketone, and SR1129.

41. The composite electrophoretic separation medium according to claim 35, wherein the photoinitiator is 1-hydroxy-cyclohexyl-phenyl-ketone (1-HCPK).

42. The composite electrophoretic separation medium according to claim 35, wherein at least the resolving portion further comprises an amine transfer agent.

43. The composite electrophoretic separation medium according to claim 42, wherein the amine transfer agent is triethylamine.

44. The composite electrophoretic separation medium according to claim 35, further comprising a denaturant.

45. The composite electrophoretic separation medium according to claim 44, wherein the denaturant is SDS.

46. The composite electrophoretic separation medium according to claim 35, further comprising a plurality of wells in at least the resolving portion, wherein the wells are configured such that a sample can be loaded in each well.

47. An electrophoretic system comprising:
a sealed cassette comprising:
a first gel portion and a second gel portion, said first and second gel portions comprising agarose, linear polyacrylamide, and a buffering system; and
a resolving portion positioned between the first and second gel portions, said resolving portion comprising agarose, polyacrylamide and a buffering system, said resolving portion lacking linear polyacrylamide; and
a first electrode in contact with the first gel portion; and
a second electrode in contact with the second gel portion.

48. The electrophoretic system according to claim 47, wherein the resolving portion further comprises a plurality of wells in at least the resolving portion, wherein the wells are configured such that a sample can be loaded in each well.

49. The electrophoretic system according to claim 48, wherein the cassette comprises a plurality of apertures in the cassette.

50. The electrophoretic system according to claim 49, wherein the apertures in the cassette are in register with the wells in the resolving portion.

51. The electrophoretic system according to claim 47, wherein the concentration of agarose in the first gel portion, the second gel portion or the resolving portion is less than about 5%.

52. The electrophoretic system according to claim 47, wherein the concentration of agarose in the first gel portion, the second gel portion or the resolving portion is between 1% to 2%.

53. The electrophoretic system according to claim 47, wherein the concentration of polyacrylamide in the resolving portion is between about 1% to about 8.5%.

54. The electrophoretic system according to claim 47, wherein the buffer system comprises at least one of bistris, tricine, BES (2-[bis(2-hydroxyethyl)am-ino]ethanesulfonic acid), MOPS (3-[N-Morpholino]propanesulfonic acid), or MES (2-(N-Morpholino)ethanesulfonic acid).

55. The electrophoretic system according to claim 47, wherein the photoinitiator is selected from the list 1-hydroxy-cyclohexyl-phenyl-ketone (1-HCPK), 2,2-dimethoxy-2-phenylacetophenone, 2-methyl-1-[4-(methylthio)phenyl]-2-(4-morpholinyl)-1-propanone, 2-hydroxy-2-methyl-1-phenyl-1-propanone, 4-(2-hydroxyethoxy)phenyl]-2-(hydroxy-2-propyl)ketone, and SR1129.

56. The electrophoretic system according to claim 47, wherein the photoinitiator is 1-hydroxy-cyclohexyl-phenyl-ketone (1-HCPK).

57. The electrophoretic system according to claim 47, wherein at least the resolving portion further comprises an amine transfer agent.

58. The electrophoretic system according to claim 57, wherein the amine transfer agent is triethylamine.

59. The electrophoretic system according to claim 47, further comprising a denaturant in the electrophoretic separation medium.

60. The electrophoretic system medium according to claim 59, wherein the denaturant is SDS.
